(19) United States
(12) Patent Application Publication
(10) Pub. No.: US 2026/0115381 A1
(43) Pub. Date: Apr. 30, 2026

(54) APPARATUS AND METHOD FOR OCCLUSION DETECTION USING PUMP OPERATION MEASUREMENT AND PUMP MOTOR CURRENT

(71) Applicant: Becton, Dickinson and Company, Franklin Lakes, NJ (US)

(72) Inventors: Simon O'Reilly, Balrath, Meath (IE); Scott Stewart, Pittsburgh, PA (US)

(73) Assignee: Becton, Dickinson and Company, Franklin Lakes, NJ (US)

(21) Appl. No.: 19/429,414

(22) Filed: Dec. 22, 2025

Related U.S. Application Data
(63) Continuation-in-part of application No. 18/962,131, filed on Nov. 27, 2024.
Application No. 18/962,131 is a continuation of application No. 16/967,330, now Pat. No. 12,156,989.
(63) Continuation-in-part of application No. 18/255,293.
(60) Provisional application No. 62/626,909, filed on Feb. 6, 2018.
(60) Provisional application No. 62/663,682, filed on Apr. 27, 2018.
(60) Provisional application No. 62/764,998, filed on Aug. 20, 2018.
(60) Provisional application No. 63/125,508, filed on Dec. 15, 2020.

(51) Int. Cl. A61M 5/168; A61M 5/142

(52) U.S. Cl. CPC A61M 5/16831; A61M 5/14212; A61M 2005/14208; A61M 2005/16863; A61M 2205/18; A61M 2205/3331

(57) ABSTRACT

A technical solution for monitoring operation of a medical delivery device such as a wearable patch pump for occlusion is provided that employs sensing pump motor current during at least one of a dispense stroke and an aspirate in a pump cycle. The solution can be implemented alone or in combination with other occlusion sensing methods that use one or more of pump measurement data such as pump stroke duration (e.g., duration of aspirate stroke or dispense stroke in a rotational metering-type pump or a reciprocating-type pump), end-stop or limit switch activation, and duration difference between aspirate and dispense strokes to detect occlusion. Accuracy of occlusion detection based on measured data in a fluid delivery device is improved by using dead band normalization of loaded measured data to unloaded measured data.

This application is a continuation-in-part of U.S. patent application Ser. No. 18/962,131, filed Nov. 27, 2024, which is a continuation of U.S. patent application Ser. No. 16/967,330, filed Aug. 4, 2020 and issued as U.S. Pat. No. 12,156,989 on Dec. 3, 2024, which is based on PCT Application No. PCT/US2019/015622, filed Jan. 29, 2019, which claims the benefit of U.S. Provisional Application Ser. No. 62/626,909, filed Feb. 6, 2018, U.S. Provisional Application Ser. No. 62/663,682, filed Apr. 27, 2018 and U.S. Provisional Application Ser. No. 62/764,998, filed Aug. 20, 2018, and a continuation-in-part of U.S. patent application Ser. No. 18/255,293, filed May 31, 2023, which is based on PCT Application No. PCT/US2021/062562, filed Dec. 9, 2021, which claims the benefit of U.S. Provisional Application Ser. No. 63/125,508, filed Dec. 15, 2020, the entire contents of each of which are incorporated herein by reference.

### BACKGROUND

### Field

The present invention relates to systems, methods and apparatuses for occlusion detection. Illustrative embodiments of the present invention relate to occlusion detection using pump motor current and/or a pump operation timing parameter such as aspirate or dispense stroke duration or end-stop switch activation in a rotational metering or reciprocating pump to obviate adding an additional pressure sensing component.

### Description of Related Art

Bolus and/or infusion pump therapy generally requires an infusion cannula, typically in the form of an infusion needle and/or a flexible catheter, that pierces the patient's skin and through which, infusion of a medicament takes place. Infusion pump therapy offers the advantages of continuous infusion, precision dosing, and programmable delivery schedules.

To facilitate drug or medicament delivery therapy, there are generally two types of pumps, namely, conventional pumps and patch pumps. Conventional pumps require the use of a disposable component, typically referred to as an infusion set, tubing set or pump set, which conveys a medicament from a reservoir within the pump into the skin of the user. The infusion set consists of a pump connector, a length of tubing, and a hub or base from which a cannula in the form of a hollow metal infusion needle or flexible plastic catheter extends. The base typically has an adhesive that retains the base on the skin surface of a user during use of the pump. The cannula can be inserted onto the skin manually or with the aid of a manual or automatic insertion device. The insertion device may be a separate unit required by the user.

Unlike a conventional infusion pump and infusion set combination, a patch pump is an integrated device that combines most or all of the fluidic components, including the fluid reservoir, pumping mechanism and mechanism for inserting the cannula, in a single housing that is adhesively attached to an infusion site on the patient's skin, and does not require the use of a separate infusion or tubing set. A patch pump containing a medicament adheres to the patient's skin and delivers the medicament over a period of time or at a selected time via an integrated subcutaneous cannula. Some patch pumps may wirelessly communicate with a separate controller device, while others are completely self-contained. Such devices can be replaced on a frequent basis, such as every three days, when the medicament reservoir is exhausted or a component malfunctions, or when complications may otherwise occur, such as restriction in the cannula or the infusion site or other occlusion.

Anomalies or dysfunctions such as leaks, occlusions or presence of air bubbles in a fluid path can occur in an infusion pump and are not necessarily noticeable to the user. Detection of a dysfunction such as a partial or total occlusion along a fluid path in an infusion pump can be desirable to maintain accurately controlled medication delivery and to advise the user to discontinue use of a malfunctioning infusion device. A typical solution for occlusion detection is to place a pressure sensor in the infusion pump system and report occlusion when the pressure is above a certain threshold. Adding a pressure sensor, however, increases the complexity of the system (e.g., increases mechanical, electrical, and/or software complexity), increases system power consumption, and increases the cost of the infusion pump.

For medical devices such as a wearable medication delivery pump, where some or all of the components are disposable for ease of use and cost effectiveness, adding another component such as a pressure sensor and related increased cost and complexity to the medical device is undesirable. A need therefore exists for accurate occlusion detection without adding infusion pump components and thereby increasing infusion pump complexity and cost.

### SUMMARY

The above and other problems are overcome, and additional advantages are realized, by illustrative embodiments of the present invention.

It is an aspect of illustrative embodiments to provide an infusion device with integral occlusion sensing. For example, a wearable infusion patch pump in accordance with an illustrative embodiment is provided that comprises: a pump comprising a chamber configured with at least one port to receive fluid into the chamber from a reservoir and to pass the fluid out of the chamber, and a pumping mechanism comprising a gearbox assembly operated by a motor to extend a piston in the chamber to control dispensing of a volume of the fluid from the chamber during a dispense stroke; a current sensing device configured to detect motor current corresponding to current drawn by the motor during each the dispense stroke performed by the pumping mechanism among a plurality of dispense strokes; and a processor. For each of the plurality of dispense strokes_{n=1, . . . , x} performed by the pumping mechanism, the processor is operated in accordance with programmed instructions to:
a processor, and for each of the plurality of dispense strokes_{n=1, . . . , x} performed by the pumping mechanism, the processor is operated in accordance with programmed instructions to:
a) determine values from the motor current corresponding to the dispense strokeₙ chosen from a dead band value (iDeadbandₙ) and a maximum piston current value (iPistonMaxₙ), the dead band value (iDeadbandₙ) corresponding to a current drawn by the motor when the pumping mechanism is moving but without causing fluid to flow with respect to the chamber such that an increase in the motor current over the dead band value (iDeadbandₙ) corresponds to a torque acting on the motor when the piston in the pumping mechanism moves against the fluid in the chamber during the dispense stroke, and the maximum piston current value (iPistonMaxₙ) corresponding to a maximum amount of current drawn by the motor during a portion of the dispense stroke wherein the piston moves against the fluid in the chamber to dispense the fluid from the at least one port;
b) determine a stroke current value (iStrokeₙ) for the dispense stroke by subtracting the dead band value (iDeadbandₙ) from the maximum piston current value (iPistonMaxₙ) corresponding to the dispense strokeₙ;
c) determine a difference (Delta_iStroke) between the stroke current value (iStrokeₙ) and a previous stroke current value (iStrokeₙ₋₁) determined for a previous one of the plurality of dispense stokes,
d) increment a counter when at least one occlusion condition is detected, the at least one occlusion condition comprising the difference (Delta_iStroke) exceeding a rapid rise threshold (iRapidRise) having a designated value corresponding to an increase in the motor current that indicates an unwanted occlusion occurring in the pump; and
e) terminating dispensing of the fluid from the chamber when the counter reaches a value T≥2.

In accordance with aspects of illustrative embodiments of the present disclosure, the current sensing device comprises a resistor connected in series with the motor, and the processor is configured to calculate the motor current using a voltage measured across the resistor.

In accordance with aspects of illustrative embodiments of the present disclosure, the dispense stroke is characterized by a stroke duration comprising a first time period corresponding to a dead band period during which the pumping mechanism is moving but without causing fluid to flow with respect to the chamber, and a second time period that follows the first time period and during which the pumping mechanism can cause the fluid to flow from the chamber.

In accordance with aspects of illustrative embodiments of the present disclosure, the processor determines the dead band value (iDeadbandₙ) from calculating the motor current using a voltage measured across the resistor during the first time period.

In accordance with aspects of illustrative embodiments of the present disclosure, wherein the processor determines the maximum piston current value (iPistonMaxₙ) from calculating the motor current using a voltage measured across the resistor during the second time period.

In accordance with aspects of illustrative embodiments of the present disclosure, the processor determines the maximum piston current value (iPistonMaxₙ) from calculating a maximum value of the motor current during a beginning portion of the second time period.

In accordance with aspects of illustrative embodiments of the present disclosure, the pumping mechanism is configured to operate for at least a designated total number (X) of dispense strokes_{n=1, . . . , x} to deliver the fluid from the chamber, and the stroke current value (iStrokeₙ) is determined using a first calculation during a designated initial number (n=1, . . . , Y) of the dispense strokes, and is determined using a second calculation different from the first calculation during a remaining number (n=Y+1, . . . , X) of the dispense strokes.

In accordance with aspects of illustrative embodiments of the present disclosure, the first calculation corresponds to the processor subtracting the dead band value (iDeadbandₙ) from the maximum piston current value (iPistonMaxₙ) corresponding to the dispense stroke_{n=1, . . . , Y}.

In accordance with aspects of illustrative embodiments of the present disclosure, the second calculation comprises the processor determining a mean value of the dead band value (iDeadbandₙ) for each of the designated initial number (n=1, . . . , Y) of the dispense strokes, and using the first calculation when the dead band value for the dispense stroke is greater than or equal to the mean value and using the second calculation when the dead band value for the dispense stroke is less than the mean value, the second calculation corresponding to subtracting the mean value from the maximum piston current value corresponding to the dispense stroke_{n=Y+1, . . . , X}.

In accordance with aspects of illustrative embodiments of the present disclosure, the processor is further configured to operate in accordance with programmed instructions to determine a current baseline value (iBaseline), and to use the current baseline value (iBaseline) instead of the stroke current value (iStrokeₙ) for dispense stroke to determine the difference (Delta_iStroke) when the stroke current value (iStrokeₙ) is less than the baseline value (iBaseline).

In accordance with aspects of illustrative embodiments of the present disclosure, the processor is further configured to operate in accordance with programmed instructions to determine the baseline value (iBaseline) during an initial operation related to the pump chosen from manufacturing of the patch pump, assembling the patch pump, initializing the patch pump, and a priming operation by the pump.

In accordance with aspects of illustrative embodiments of the present disclosure, the processor is further configured to operate in accordance with programmed instructions to calculate the baseline value (iBaseline) using a mean value of motor current corresponding to a predetermined number of the dispense stroke previously occurring during the initial operation and precluding the motor current from the mean value when the motor current differs from the mean value by a designated amount.

In accordance with aspects of illustrative embodiments of the present disclosure, the processor is further configured to operate in accordance with programmed instructions to determine an acceptable backpressure threshold value (iDelta_Threshold) that corresponds to the motor current occurring while the piston is operating against patient-related backpressure that occurs when the pump is delivering the fluid into a patient and is less than an unwanted occlusion being detected by the processor. The at least one occlusion condition further comprises the stroke current value (iStrokeₙ) exceeding an occlusion threshold value (iThreshold) corresponding to a sum of the current baseline value (iBaseline) and the acceptable backpressure threshold value (iDelta_Threshold).

In accordance with aspects of illustrative embodiments of the present disclosure, the rapid rise threshold (iRapidRise) is determined experimentally during operation of the pump to be a value of the motor current at which an unwanted occlusion is not occurring in the pump.

Additional and/or other aspects and advantages of embodiments of the present invention will be set forth in the description that follows, or will be apparent from the description, or may be learned by practice of the invention. The present invention may comprise devices and methods for operating same having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The above and/or other aspects and advantages of embodiments of the invention will be more readily appreciated from the following detailed description, taken in conjunction with the accompanying drawings, of which:

FIGS. 1 and 2 are partial, perspective views of example pump components in an example medication delivery device that operates in accordance with an occlusion; detection algorithm in accordance with an illustrative embodiment of the present invention;

FIGS. 3A and 3B are perspective views of pump components of FIGS. 1 and 2 in an example medication delivery device arranged, respectively, in accordance with a ready to dispense stage of operation and a ready to aspirate stage of operation;

FIG. 3C is a perspective view of components in an example medication delivery device comprising example pump components of FIGS. 1 and 2 and associated electronic circuits on a printed circuit board;

FIG. 3D is a partial perspective view of an example motor and gearbox assembly configured to cooperate with example pump components of FIGS. 1 and 2;

FIG. 4 is a block diagram of components in an example medication delivery device;

FIGS. 5A and 5B are, respectively, diagrams illustrating pump duration times for a plurality of aspirate operations and a plurality of dispense operations of an example medication delivery device under normal operating conditions;

FIGS. 6A and 6B are, respectively, diagrams illustrating pump duration times for a plurality of aspirate operations and a plurality of dispense operations of the same type of medication delivery device used to generate FIGS. 5A and 5B but under occluded operating conditions;

FIG. 7 is a flow chart of illustrative operations of an example medication delivery device that operates in accordance with an occlusion detection algorithm employing stroke duration criteria in accordance with an illustrative embodiment of the present invention;

FIGS. 8A and 8B depict, respectively, example end-stop or limit switch activation data during normal and occluded operation of an illustrative pump;

FIG. 9 is a flow chart of illustrative operations of an example medication delivery device that operates in accordance with an occlusion detection algorithm employing end-stop or limit switch activation duration criteria in accordance with an illustrative embodiment of the present invention;

FIG. 10 depicts example pump measurement data indicating a short dispense stoke duration (e.g., such as when the pump piston is not able to move during an occlusion);

FIG. 11 depicts example pump measurement data indicating an extended end-stop or limit switch activation duration (e.g., such as when pumping back to the pump reservoir occurs due to an occlusion);

FIGS. 12A, 12B, 12C and 12D depict pump measurement data from respective pumps indicating long dispense stroke duration relative to aspirate stroke duration (e.g., such as when leaking occurs due to an occlusion);

FIG. 13 is a flow chart of illustrative operations of an example medication delivery device that operates in accordance with an occlusion detection algorithm employing leak detection criteria in accordance with an illustrative embodiment of the present invention;

FIG. 14 is a flow chart of illustrative operations of an example medication delivery device that operates in accordance with an occlusion detection algorithm employing a combination of criteria in accordance with an illustrative embodiment of the present invention;

FIG. 15 is a schematic diagram of a medication delivery device pump motor having a current sensor in accordance with an illustrative embodiment of the present invention;

FIG. 16 is a flow chart of illustrative operations of an example medication delivery device that operates in accordance with an occlusion detection algorithm employing pump motor current criteria in accordance with an illustrative embodiment of the present invention;

FIGS. 17A, 17B, 17C, 17D and 17E depict pump measurement data from respective example delivery devices indicating motor current during a dispense stroke before and after occlusion;

FIGS. 18A, 18B, 18C, 18D and 18E depict average motor current for a selected time period for respective example delivery devices;

FIG. 19 is a flow chart of illustrative operations of an example medication delivery device that operates in accordance with an occlusion detection algorithm employing a combination of criteria with pump motor current criteria in accordance with an illustrative embodiment of the present invention;

FIGS. 20A and 20B depict, respectively, raw and filtered data (e.g., motor current) from an example fluid delivery device during aspirate and dispense strokes;

FIG. 20C depicts filtered measured data (e.g., motor current) from an example fluid delivery device during dispensing and variance at different pressures;

FIG. 20D depicts measured data (e.g., motor current) during operation of an example fluid delivery device with its drive mechanism component(s) moving fluid and not moving fluid to illustrate a dead band normalization region in the data;

FIG. 21 depicts measured data (e.g., motor current) from an example fluid delivery device during a dispense operation and data from region therein identified for dead band normalization;

FIG. 22 is a flow chart of illustrative operations of an example fluid delivery device performing a dispense operation with dead band normalization of measured data in accordance with an illustrative embodiment; and

FIG. 23 is a flow chart of illustrative operations of an example medication delivery device that operates in accordance with an occlusion detection algorithm employing pump motor current criteria in accordance with another illustrative embodiment of the present invention.

Throughout the drawing figures, like reference numbers will be understood to refer to like elements, features and structures.

### DETAILED DESCRIPTION OF ILLUSTRATIVE EMBODIMENTS

As will be appreciated by one skilled in the art, there are numerous ways of carrying out the examples, improvements, and arrangements of a fluid delivery device in accordance with embodiments described herein. Although reference will be made to the illustrative embodiments depicted in the drawings and the following descriptions, the embodiments disclosed herein are not meant to be exhaustive of the various alternative designs and embodiments that are encompassed by the disclosed technical solutions, and those skilled in the art will readily appreciate that various modifications may be made, and various combinations can be made with departing from the scope of the disclosed technical solutions.

Illustrative embodiments can be employed with any type of infusion pump that works on the principles of filling a chamber (e.g., with liquid medication from a reservoir) in one stage and then emptying the fluid from the chamber (e.g., to a delivery device such as a cannula deployed in a patient) in another stage, or at least emptying fluid from a chamber. For example, for some example embodiments herein, a reciprocating plunger-type pump or a rotational metering-type pump can be used, and for other example embodiments herein, any of a reciprocating plunger-type pump, or a rotational metering-type pump, or a syringe-type pump having a barrel as the chamber, can be used. In either case of a reciprocating plunger-type pump or a rotational metering-type pump, a piston or plunger is retracted from a chamber to aspirate or draw medication into the chamber and allow the chamber to fill with a volume of medication (e.g., from a reservoir or cartridge of medication into an inlet port). The piston or plunger is then re-inserted into the chamber to dispense or discharge a volume of the medication from the chamber (e.g., via an outlet port) to a fluid pathway extending between the pump and a cannula in the patient. For a syringe-type pump, a plunger and drive mechanism operate to selectively drive the plunger to dispense fluid from the barrel. An example syringe-type pump is described in commonly owned International PCT patent publication no. WO 2022/132555, the content of which is incorporated herein by reference in its entirety.

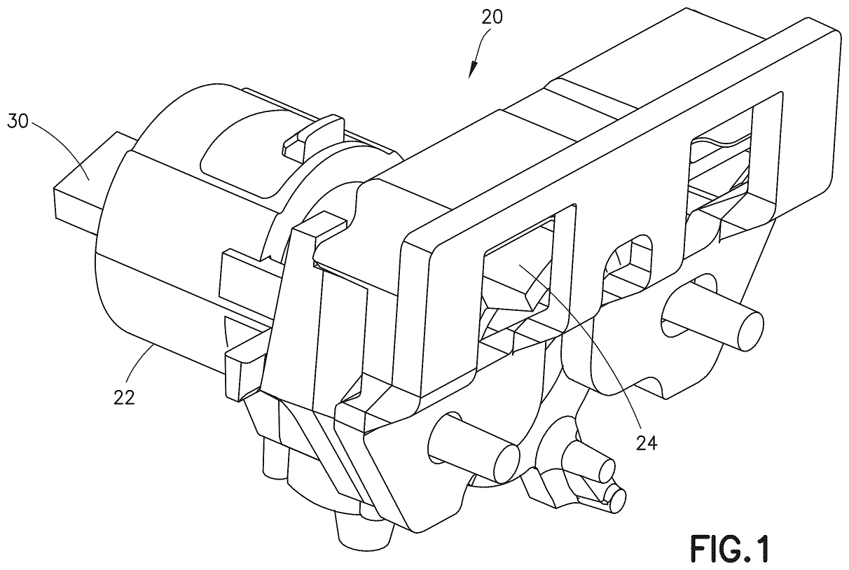

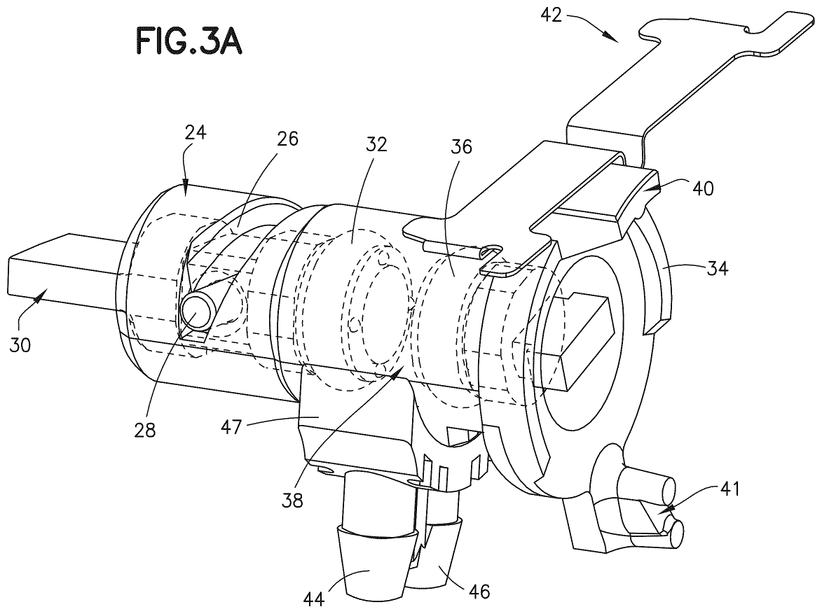

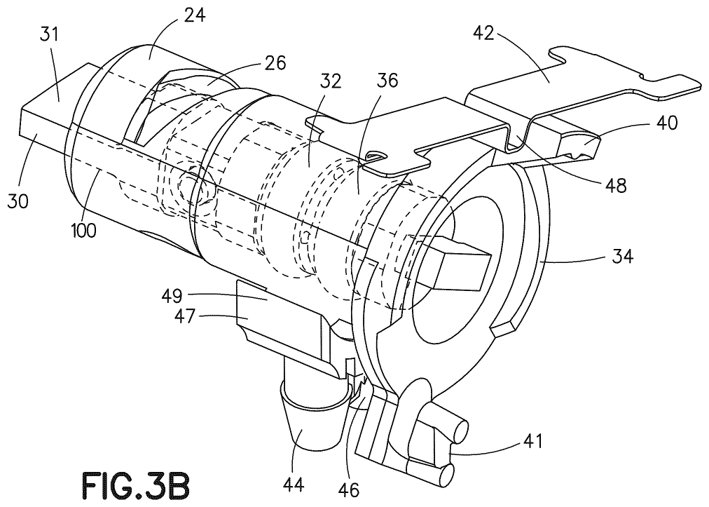

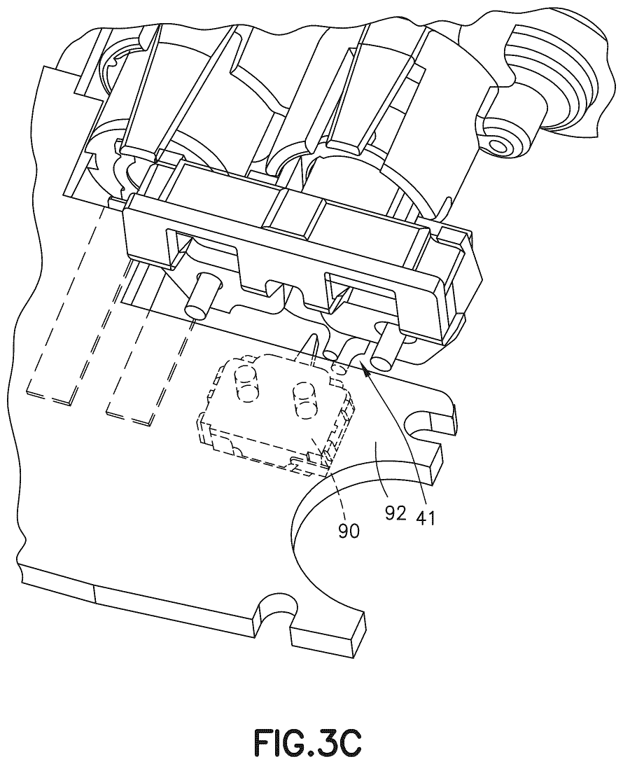

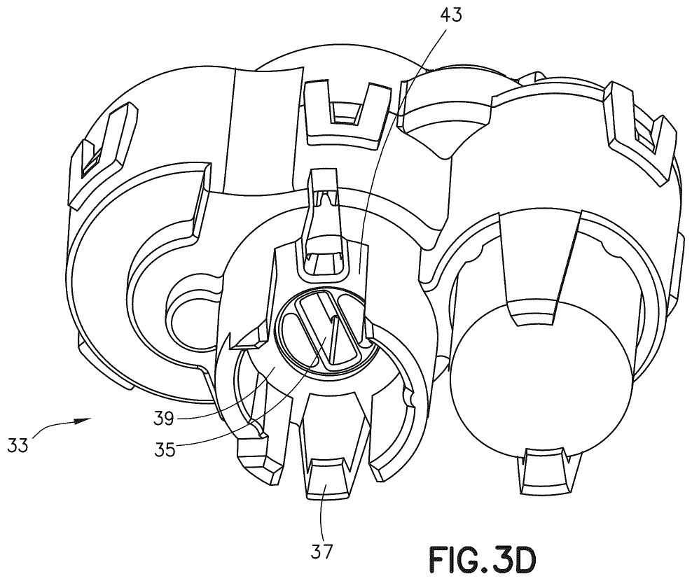

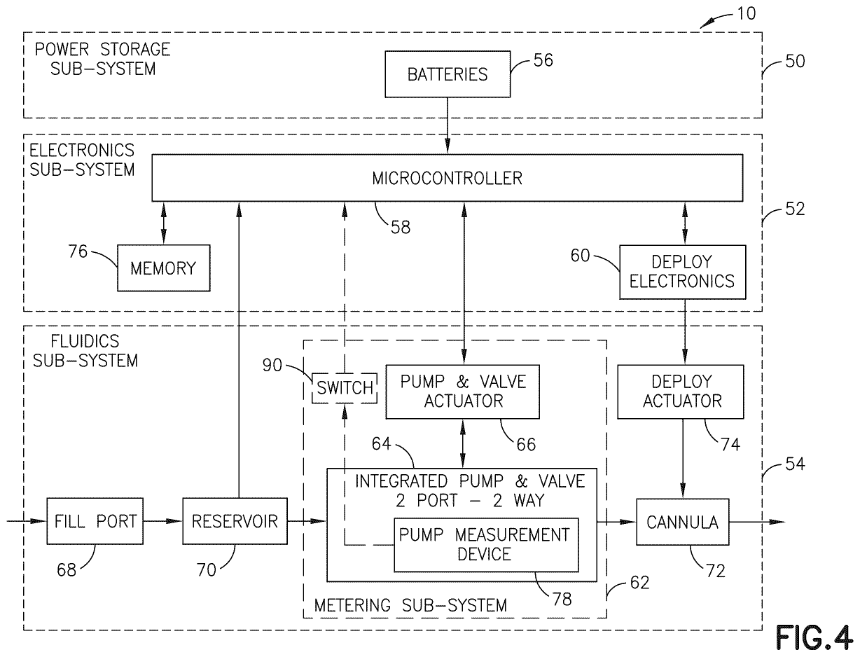

For illustrative purposes, reference is made to an example rotational metering-type pump described in commonly owned WO 2015/157174, the content of which is incorporated herein by reference in its entirety. With reference to FIGS. 1, 2, 3A, 3B, 3C and 3D, an example infusion pump (e.g., a wearable medication delivery device such as a patch pump) comprises a pump assembly 20 which can be connected to a DC motor 66 (FIG. 4) and gearbox assembly 33 (FIG. 3D) to rotate a sleeve 24 in a pump manifold 22. A helical groove 26 is provided on the sleeve. A coupling pin 28 connected to a piston 30 translates along the helical groove to guide the retraction and insertion of the piston 30 within the sleeve 24, respectively, as the sleeve 24 rotates in one direction and then rotates in the opposite direction. The sleeve has an end plug 34. Two seals 32, 36 on the respective ends of the piston and end plug that are interior to the sleeve 24 define a cavity or chamber 38 when the piston 30 is retracted, as depicted in FIG. 3A, following an aspirate stroke and therefore ready to dispense. The volume of the chamber 38 therefore changes depending on the degree of retraction of the piston 30. The volume of the chamber 38 is negligible or essentially zero when the piston 30 is fully inserted and the seals 32, 36 are substantially in contact with each other following a dispense stroke, as depicted in FIG. 3B, and therefore ready to aspirate. Two ports 44, 46 are provided relative to the pump manifold 22, including an inlet port 44 through which medication can flow from a reservoir 70 (FIG. 4) for the pump 64 (FIG. 4), and an outlet port 46 through which the medication that has been drawn into the chamber 38 (e.g., by retraction of the piston 30 during an aspirate stage of operation) can be dispensed from the chamber 38 to, for example, a fluid path to a cannula 72 (FIG. 4) in the patient by re-insertion of the piston 30 into the chamber 38.

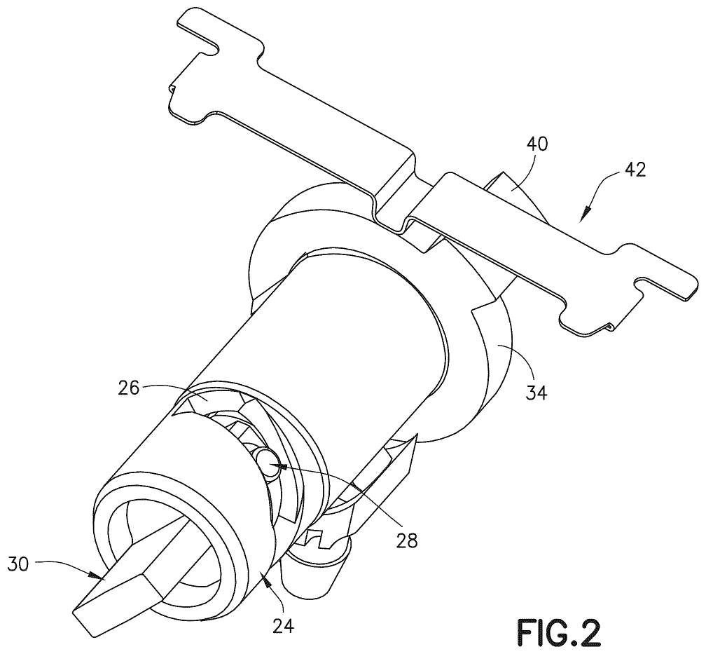

With continued reference to FIGS. 1, 2, 3A, 3B, 3C and 3D, the sleeve 24 can be provided with an aperture (not shown) that aligns with the outlet port 46 or the inlet port 44 (i.e., depending on the degree of rotation of the sleeve 24 and therefore the degree of translation of the piston 30) to permit the medication in the chamber 38 to flow through the corresponding one of the ports 44, 46. A pump measurement device 78 (FIG. 4) such as a sleeve rotational limit switch can be provided which has, for example, an interlock 42 and one or more detents 40 on the sleeve 24 or its end plug 34 that cooperate with the interlock 42. The interlock 42 can be mounted to the manifold 22 at each end thereof. The detent 40 at the end face of sleeve 24 is adjacent to a bump 48 of the interlock 42 when the pump 64 is in a first position whereby a side hole in the sleeve 24 is aligned with the inlet port 44 to receive fluid from the reservoir 70 into the chamber 38. Under certain conditions, such as back pressure, it is possible that friction between the piston 30 and the sleeve 24 is sufficient to cause the sleeve 24 to rotate before the piston 30 and coupling pin 28 reach either end of the helical groove 26. This could result in an incomplete volume of liquid being pumped per stroke. In order to prevent this situation, the interlock 42 prevents the sleeve 24 from rotating until the torque passes a predetermined threshold, as shown in FIG. 3A. This ensures that piston 30 fully rotates within the sleeve until the coupling pin reaches the end of the helical groove 26. Once the coupling pin 28 hits the end of the helical groove 26, further movement by the DC motor and gearbox assembly 33 or other type of pump and valve actuator 66 (FIG. 4) increases torque on the sleeve 24 beyond the threshold, causing the interlock 42 to flex and permit the detent 40 to pass by the bump 48. At the completion of rotation of the sleeve 24 such that its side hole is oriented with the cannula 72 or outlet port 46, the detent 40 moves past the bump 48 in the interlock 42, as shown in FIG. 3B. Another sleeve feature 41 can be provided to engage an electrical switch (e.g., an end-stop switch 90 provided on a printed circuit board 92 and disposed relative to the sleeve and/or end plug 34 to cooperate with the pump measurement device 78 as shown in FIG. 3C). FIG. 3D is a partial perspective view of an example motor and gearbox assembly 33 configured to cooperate with example pump components of FIGS. 1 and 2 and is described more below in connection with providing an example interface for dead band normalization.

FIG. 4 is an illustrative system diagram that illustrates example components in an example medication delivery device 10 having an infusion pump such as the pump of FIGS. 1, 2, 3A, 3B, 3C and 3D. The medication delivery device 10 can include an electronics sub-system 52 for controlling operations of components in a fluidics sub-system 54 such as the pump 64 and an insertion mechanism 74 for deploying a cannula 72 for insertion into an infusion site on a patient's skin. A power storage sub-system 50 can include batteries 56, for example, for providing power to components in the electronics and fluidics sub-systems 52 and 54. The fluidics sub-system 54 can comprise, for example, an optional fill port 68 for filling a reservoir 70 (e.g., with medication), although the medication delivery device 10 can be optionally shipped from a manufacture having its reservoir already filled. The fluidics sub-system 54 also has a metering sub-system 62 comprising the pump 64 and a pump actuator 66. As described above, the pump 64 can have two ports 44, 46 and related valve sub-assembly that controls when fluid enters and leaves a pump chamber 38 via the respective ports 44, 46. One of the ports is an inlet port 44 through which fluid such as liquid medication flows from the reservoir 70 into the pump 64 as the result of a pump intake or pull stroke on a pump plunger or piston 30, for example. The other port is an outlet port 46 through which the fluid leaves the pump's chamber 38 and flows toward a cannula 72 for administration to a patient pump as the result of a pump discharge or push stroke on the pump plunger or piston 30. The pump actuator 66 can be a DC motor and gearbox assembly 33 or other pump driving mechanism for controlling the plunger or piston 30 and other related pump parts such as a sleeve 24 that may rotate relative to the translational movement of the pump piston 30. The microcontroller 58 can be provided with an integrated or separate memory device having computer software instructions to actuate, for example, rotation of the sleeve 24 in a selected direction, translational or axial movement of a piston 30 in the sleeve 24 for an aspirate or dispense stroke, and optionally the rotation of the sleeve 24 and piston 30 together during a valve state change as described in the above-referenced WO 2015/157174. As described below, an occlusion detection algorithm in accordance with illustrative embodiment can be provided to the microcontroller 58 to monitor pump measurements and detect when occlusion operating condition occurs relative to the infusion pump.

Regardless of the type of pump mechanism 64 used to aspirate a controlled volume of medication into a pump chamber 38 and to dispense a controlled volume of medication from the pump chamber, the pump 64 has associated therewith an expected pump duration for one or both of the aspirate and dispenses stages or strokes which can be attributed to the pump characteristics. For example, in the illustrative pump assembly 20 shown in FIGS. 1, 2, 3A, 3B, 3C and 3D, the pump's duration for aspirating medication into the chamber and for dispensing the medication from the chamber 38 is affected by such pump characteristics as the internal volume of the pump chamber 38, the length or distance of a pump piston stroke, characteristics of port seals provided at the inlet and output ports 44, 46, etc., When the pump pressure is within a designated relative normal range for operation, the pump duration for filling the chamber 38 with a designated amount of fluid (e.g., a desired dosage) and for discharging the designated amount of fluid from the chamber can be determined and used as a baseline for monitoring the pump 64 for normal operating conditions and for determining when an abnormal operating condition has arisen such as due to a leakage of fluid from the pump chamber or an occlusion in the pump fluid path whereby, in either scenario, the designated amount of fluid (e.g., a desired dosage) cannot be delivered from the chamber via a dispense stroke. This can be undesirable since the patient will not be receiving the desired dosage.

As stated above, a typical solution for occlusion detection is to place an additional pressure sensor in the pump control system and report occlusion when the pressure is above a certain threshold. Adding a pressure sensor, however, has the drawbacks of increasing the complexity of the system (e.g., mechanical, electrical, and/or software complexity), increasing system power consumption, and/or increasing pump cost. These drawbacks can be particularly disadvantageous to a wearable pump design wherein all or part of the pump is intended to be disposable once the reservoir 70 is emptied or the pump 64 has been used to a selected amount of time and/or to deliver a selected amount of medication.

In accordance with illustrative embodiments, occlusion detection is accomplished without an additional component such as an occlusion sensor deployed upstream or downstream of the pump 64. When a microcontroller 58 or other processing device for controlling pump operation already performs pump duration measurements for normal operations such as for one or both of aspirate strokes and dispense strokes, the microcontroller 58 can be further controlled to determine when a pump duration measurement is outside a designated range of normal operating conditions and therefore indicates an occlusion, and generate an indication of detected occlusion. The pump 64 and/or the entire medication delivery device 10 can therefore, in turn, be replaced or repaired, thereby ensuring that the patient is receiving the full intended dosage that is provided under normal operating conditions.

When pump duration measurement is implemented for pump operation, occlusion detection can be achieved by adding to the computer software instructions of the microcontroller 58, or a remote device that controls the medication delivery device 10, such operations as monitoring pump duration and determining when a designated pump duration threshold or other criteria for normal pump operating conditions is not met. Thus, occlusion detection is implemented via a software solution, and no hardware changes to the pump are needed. As will be described below, a clear distinction of pump duration exists between the normal and occluded pumps; therefore, the false alarm rate and miss rate are quite low. Therefore, an occlusion detection algorithm configured in accordance with aspects of illustrative embodiments is able to provide reliable occlusion detection results.

Determining a pump duration threshold value or range of values or other metric that indicates occlusion can be performed empirically for a selected type of pump 64, for example. Metrics for a selected type of pump experiencing normal operational pressure can be compared with metrics for the same type of pump except that it is experiencing at least a partial or full occlusion. For example, an occlusion in a downstream path from the occluded pump 64 to its cannula 72 causes pressure in the fluid path of the pump 64 to increase over time. When pressure in the occluded pump exceeds a threshold, the occluded pump eventually begins to leak. Log files of the normal pump and the occluded pump can be generated to obtain their respective histories of pump duration information for aspirate strokes and/or dispense strokes. It is to be understood, however, that a different pump measurement besides pump duration (i.e., duration of an aspirate stroke or a dispense stroke) such as pump motor current can be used to determine differences in pump operations during normal and occluded operating conditions and to determine a threshold for monitoring pump operations and distinguishing between a normal operating condition and an occluded operating condition. For example, as described below, a prolonged end-of-stroke switch activation or significant difference in the respective durations of an aspirate stroke and a dispense stroke can be used to detect the occurrence of an occlusion with or without use of pump motor current in accordance with different example embodiments for occlusion sensing using measured characteristics of pump operations but without having to rely on an additional component such as an occlusion sensor deployed upstream or downstream of the pump 64.

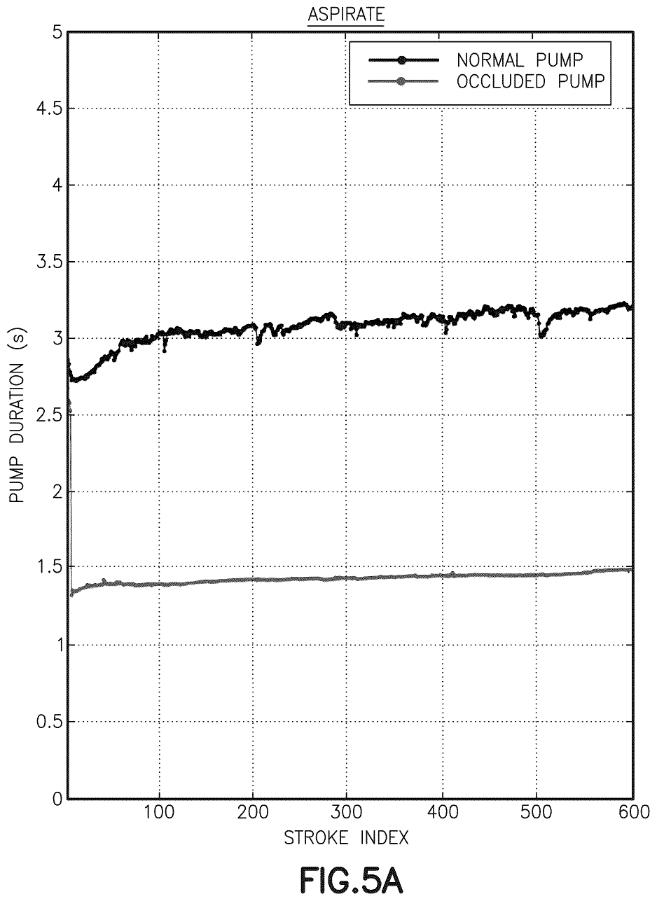

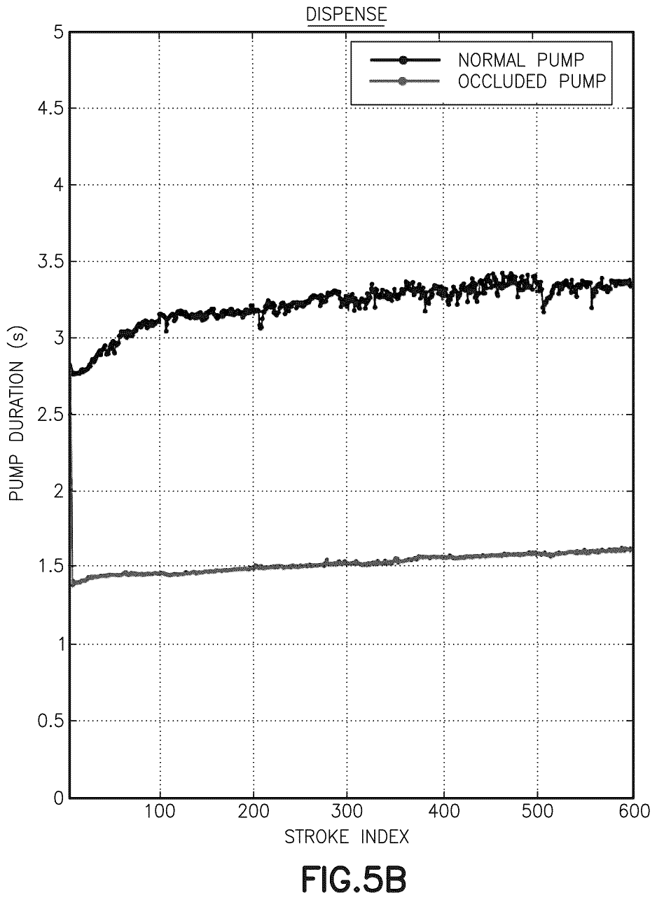

With reference to FIGS. 5A and 5B, the pump duration (e.g., approximately 1.5 seconds on average) of a pump experiencing occlusion is considerably shorter than the pump duration (e.g., on the order of 3-3.5 seconds) of the pump 64 when it is operating under normal conditions, and the phenomenon of shorter pumping duration is related to the pumping mechanism such as the piston 30, sleeve 24, interlock 42 and silicon seals on the inlet and outlet ports 44, 46 described above in connection with FIGS. 1, 2, 3A, 3B 3C and 3D. As described above, different types of pumps 64 can be improved by implementing occlusion sensing in accordance with illustrative embodiments, and different pump components can contribute to the shortened pump during an occlusion condition. The pumps 64 can be rotational metering-type pumps or reciprocating-type pumps or other type of pump that employ pulling in or aspirating fluid from an upstream reservoir, and then discharging or dispensing that fluid to a separate downstream fluid path that leads to the patient.

With reference to the example infusion pump 64 described above in connection with FIGS. 1, 2, 3A, 3B, 3C and 3D, the pump's aspirate and dispense strokes, driven by piston 30 translation within the outer plastic sleeve 24, are related to the switching of the pump 64 between the upstream and downstream fluid paths. As the piston 30 is rotated (e.g., by the DC motor and gearbox assembly that is not shown), the piston 30 translates through the sleeve 24, guided by travel of the pin 28 on the piston through a helical slot 26 in the sleeve 24. Once the piston 30 translates fully through the sleeve 24 and completes its aspiration stage or dispensing stage of fluid, it engages with the sleeve 24 directly via the pin 28 in the slot 26, and rotation of the piston 30 and sleeve 24 become coupled. This allows for the sleeve 24 to rotate between upstream and downstream fluid paths and actuate an end of stroke electrical switch 90 or other component associated with the pump measurement device 78 (FIG. 4) and provided on the pump 64 and/or in the medication delivery device 10. During normal operation, the presence of the interlock 42 prevents the piston 30 and sleeve 24 rotation from coupling prior to the piston 30 completing its translation through the sleeve 24. However, if pressure in the downstream fluid path increases beyond a threshold, the piston 30 and sleeve 24 rotation couple and allow for the sleeve 24 to pass under the interlock 42 and actuate the switch 90 (e.g., via a sleeve feature 41 associated with the pump measurement device 78) before the piston 30 has completed its translation through the sleeve. This shortens the pumping duration considerably (e.g., from between 3 and 3.5 seconds during normal conditions to less than 2 seconds during occluded conditions).

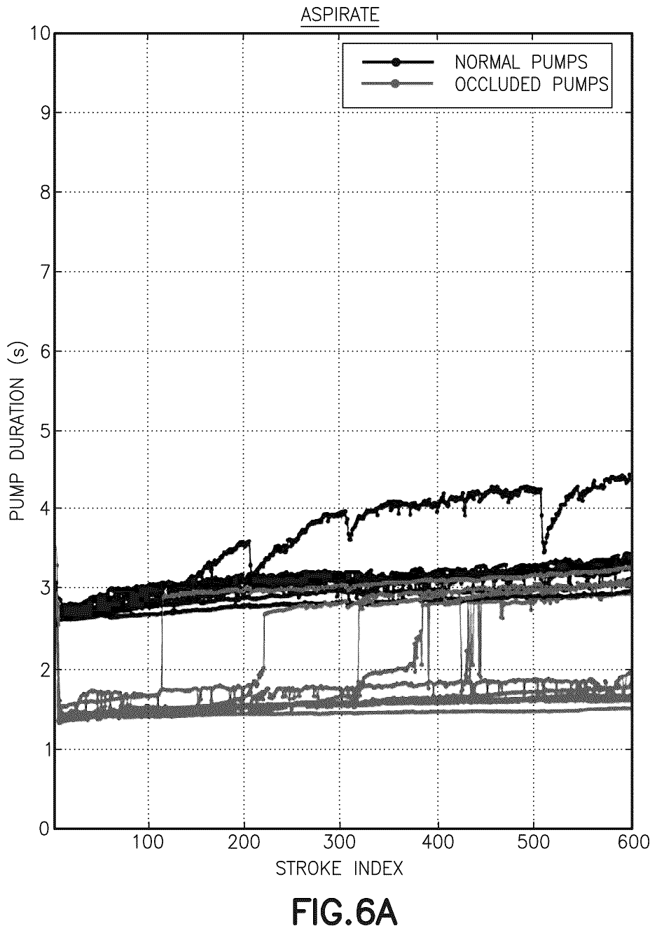

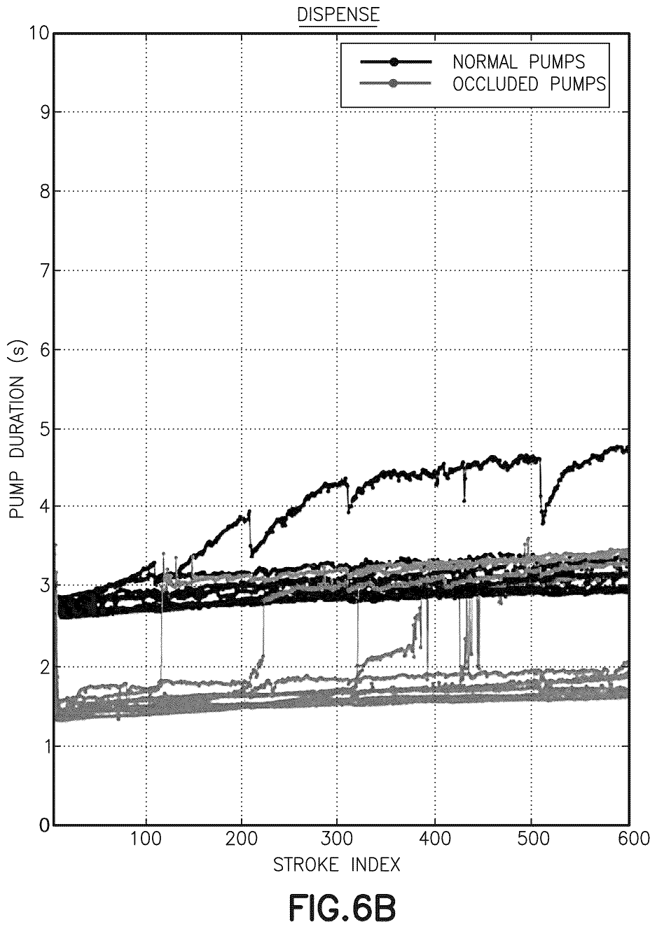

Reference is now made to FIGS. 6A and 6B which show pump duration data from a plurality of similar type pumps 64 over plural pump cycles. For example, log data from 19 pumps that completed finished 600 cycles is shown whereby 10 of the pumps operated under normal conditions, and 9 of the pumps operated under occluded conditions. It can be seen from FIGS. 6A and 6B that all of the occluded pumps had a section of pump duration less than 2 seconds. Some pump durations went back to normal, which may be due to the release of pressure from leaking at the manifold area. The clear distinction of pump duration between the normal operating pump and the pumps experiencing occlusion allows for use of an occlusion detection algorithm based on pump duration.

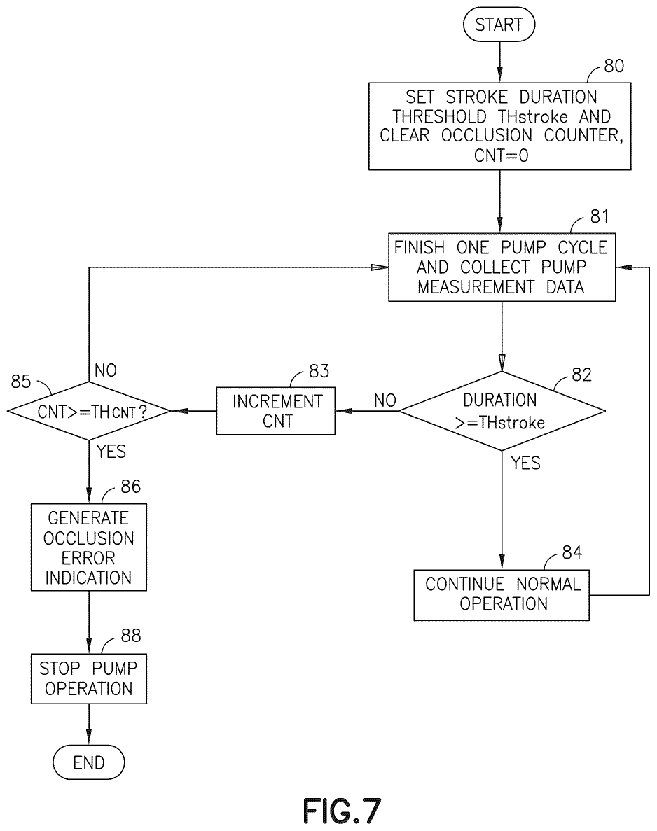

With reference to FIG. 7, an example occlusion detection process comprises setting a pump measurement threshold or metric such as a stroke duration threshold (block 80), wherein a stroke duration above the threshold indicates normal pump operation and a stroke duration below the threshold indicates occlusion. To set the threshold, pump measurement data is analyzed. For example, aspirate stroke durations and dispense stroke durations can be detected by a limit switch or other pump measurement device 78 (FIG. 4) provided to the pump. In the example pump described with reference to FIGS. 1, 2, 3A, 3B, 3C and 3D, stroke or pump durations are determined using a sleeve rotation limit switch or other pump measurement device 78. For example, a microcontroller 58 and other electronic components such as an end-stop switch 90 that cooperates with the sleeve feature 41 can be deployed on a printed circuit board (PCB) 92 associated with the pump 64 or the delivery device 10 in general. End-stop switch activation data can be collected and stored (e.g., via a memory device integral to the microcontroller 58 or implemented as a separate component on the PCB 92). The microcontroller 58 can be provided with an occlusion detection algorithm for processing the end-stop switch activation data to determine if an occlusion has occurred. In accordance with another illustrative embodiment, the end-stop switch activation data can be provided (e.g., wirelessly or via wireline connection) from the pump 64 to another device having an occlusion detection algorithm such as a hand-held remote controller for the pump 64 or a non-dedicated computing device (e.g., mobile phone, personal computer (PC), laptop or other portable computing device) provided with software or app comprising the occlusion detection algorithm.

Pump measurement data is obtained for one or more of the same type of pump operating under normal conditions, and for one or more of the same type of pump operating under occluded conditions, as illustrated above in FIGS. 5A and 5B and in FIGS. 6A and 6B. The pump measurement data for these two groups of pumps can be averaged or otherwise summarized or categorized, and then analyzed to determine the degree of difference between the pump measurements for normal operating pumps and the pump measurements for occluded pumps. A threshold or other metric is determined to be a value or a range of values with a margin(s) above and/or below which normal pump measurements will not fall. The value, or range of values, and/or margin can be designated by a user, or automatically determined based on the pump measurement data obtained from the pump. As described above, the pump measurement data is data that is generated and monitored during the course of normal pump activity and therefore is not an added operation or require an additional component that increases the complexity of the pump.

With continued reference to FIG. 7, once the pump measurement metric (e.g., stroke duration threshold) is set, the microcontroller 58 in the medication delivery device 10 is controlled by the occlusion detection algorithm to obtain pump measurement data (e.g., stroke duration data) for the pump (block 81), and to compare the stroke duration data to the pump measurement metric during various pump stages or cycles of operation such as for each pump cycle (block 82). When the stroke duration data meets the pump measurement metric (e.g., is greater or equal to a Thₛₜᵣₒₖₑ of 2 seconds for the pump 64), the pump is determined to be operating normally (block 84). When the stroke duration data fails to satisfy the pump measurement metric (e.g., is below the occlusion detection threshold (e.g., is less than a Thₛₜᵣₒₖₑ of 2 seconds for the pump 64)), then the pump is determined to be experiencing an occlusion condition. A counter is incremented (block 83) when a threshold Thₛₜᵣₒₖₑ for normal operation is not met. With reference to block 85, when the counter reaches a selected value (e.g., the counter value of 8 corresponding to 8 pump cycles wherein a threshold Thₛₜᵣₒₖₑ for normal operation is not met), then occlusion is detected. The total number of cycles during which the selected number of cycles is reached before occlusion is indicated can be designated such as 8 consecutive cycles of 8 cycles or within a designated number of cycles (e.g., 20 cycles). The microcontroller 58 can be configured by the occlusion detection algorithm to generate an optional indication of detected occlusion error (block 86), and to automatically stop operation of the pump and/or the medication delivery device 10, and/or generate an optional indication to the user to cease using the pump (block 88). If the counter, after being incremented per block 83, has not yet reached the selected counter value, then the pump measurement data continues to be collected per block 81. Since the occlusion detection algorithm is based on pump duration or other pump measurement data that has already been implemented in the pump, occlusion detection is achieved by checking pump duration or other measurement data in the software against a selected threshold or metric. Accordingly, a software-only solution is provided for occlusion detected, obviating the need for any hardware changes.

The example pump 64 described in connection with FIGS. 1, 2, 3A, 3B, 3C and 3D uses one or more on/off limit switches to determine the state of the system at the limits of rotational travel. For example, multiple stage pumps (i.e., a pump that aspirates fluid to fill a chamber during one stage and then discharges the pump chamber in the next stage) can employ an end-stop switch of some type for each stage to detect when the piston and/or a sleeve or other pump component reaches a predetermined position corresponding to a complete aspirate or dispense position. It is to be understood, however, that different mechanisms or other pump measurement device 78 can be used to determine the pump measurement (e.g., pump duration) besides an interlock 42 and sleeve rotational limit switch (e.g., end-stop switch) 90. Alternatively, the pump 64 can employ one or more optical sensors, or an encoder with optical switch to determine positions of pump components at their respective end-stop positions for complete aspiration and/or dispensing.

Thus, as described with reference to FIG. 7 and in accordance with illustrative embodiments of the invention, determination of a time needed to fill the chamber, and a time needed to discharge a desired amount of fluid from the chamber, is performed, at least the discharge times of each stroke is measured, and, when a selected number of discharge times fails to exceed a designated amount (e.g., the stroke duration shortens over a designated number of pump cycles), an indication is generated to indicate that an occlusion is detected.

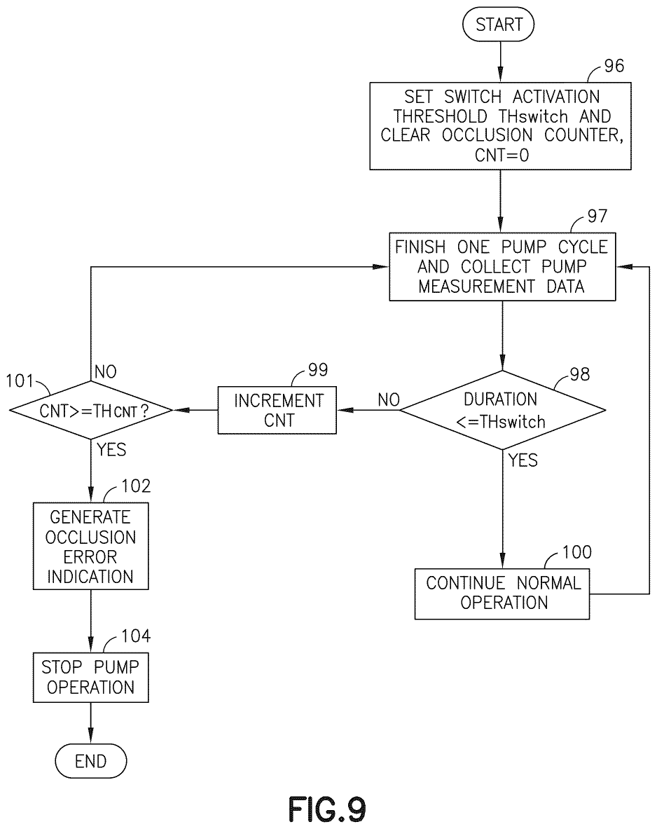

In accordance with another illustrative embodiment, occlusion detection is performed by monitoring duration of activation or triggering of a pump end-stop or limit switch, as will be described below with reference to FIG. 9. Processing monitored data related to the detected duration of activation or triggering of a pump end-stop or limit switch to determine if an occlusion in the pump 64 has occurred can be performed singly or in combination with monitoring for short pump stroke duration as described above in connection with FIG. 7.

As explained above, during normal operation, the presence of the interlock 42 prevents the piston 30 and sleeve 24 rotation from coupling prior to the piston 30 completing its translation through the sleeve 24. However, as pressure in the downstream fluid path builds (i.e., during an occlusion), the piston 30 and sleeve 24 rotation can couple prematurely; that is, the sleeve 24 rotates prematurely before an intended rotation during a valve state change, for example, when the sleeve 24 rotates at the end of a complete piston stroke and without axial motion to align its side port with a corresponding one of the ports 44, 46 during normal operation of the pump). This premature rotation coupling of the piston 30 and sleeve 24, in turn, allows for the sleeve 24 to pass under the interlock 42 and trigger the switch 78 before the piston 30 has completed its axial translation through the sleeve. This shortens the pumping duration (e.g., measured as time period or duration between pump motor startup and end-stop switch signal) considerably as explained above in connection with FIG. 7. In addition, another pump operation characteristic that can be monitored for occlusion detection is the duration that a pump measurement device 78 and its associated switch 90 is in an activated or triggered mode of operation or otherwise indicates the beginning of a state of activation.

In some instances, pump duration in an occluded pump system can remain normal and not decrease as expected; therefore, monitoring for another pump measurement parameter or characteristic increases occlusion detection accuracy. For example, while the pump sleeve 24 rotates prematurely as anticipated due to the occlusion in the pump system, and as soon as the pump sleeve opens to the upstream fluid path (and before the end-of-stroke signal from the switch 90), the piston can begin advancing and dispensing the fluid payload back into the upstream fluid path. Because both the piston 30 and sleeve 24 can rotate through their full range of angular position, the total pump operation time remains constant both with and without an occlusion. On the other hand, since the piston 30 is now rotating and translating through the sleeve 24 after the sleeve has rotated over the upstream channel, the end-stop switch 90 is now being triggered for an extended period of time. Thus, occlusion detection can comprise monitoring for prolonged or extended end-stop of limit switch activation or triggering separately, or in addition to, monitoring for shortened pump stroke duration in accordance with illustrative embodiments.

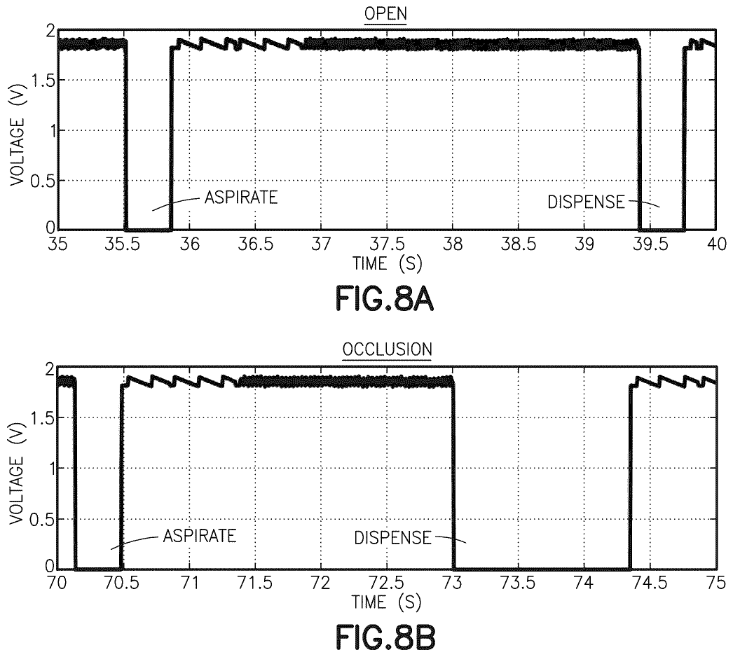

To further illustrate how activation or triggering of a pump measurement device can be prolonged as a result of an occlusion, reference is made to the example pump 64 described in accordance with the illustrative embodiment depicted in FIGS. 1, 2, 3A, 3B, 3C and 3D. During normal pump 64 operation, when the end-stop switch 90 is first hit and dragged by the pump sleeve 24 (e.g., via the sleeve feature 41 that engages with the end-stop switch 90) and therefore triggered, the end-stop switch 90 produces a drop in its end-stop switch voltage signal from 1.8 V to 0 V that is provided to the microcontroller 58. Only after the switch 90 is released (e.g., by disengagement of the sleeve feature 41) and springs back to center does the end-stop switch voltage return back to 1.8 V. When, in some instances, the side port of the sleeve 24 opens to the upstream fluid path (e.g., aligns with the input port 44) before the piston 30 has completed its axial translation and before the end-stop switch 90 has been disengaged by the sleeve feature 41, and when the pressure in the upstream fluid path is low, the piston 30 can begin to advance and translate through the sleeve 24, emptying pump contents into the upstream fluid path while the end-stop switch 90 is in a mid-trigger state. The net result is that the end-stop switch 90 activation signal (e.g., voltage drop) occurs for an extended period of time. This pump occlusion characteristic is shown in FIGS. 8A and 8B which illustrate, respectively, a normal duration of switch 90 activation (e.g., 0 volts) of less than 0.5 seconds, and an extended end-stop or limit switch 90 activation (e.g., 0 volts) of almost 1.5 seconds.

There are several reasons why some pumps 64 may exhibit a shorter overall pump duration (e.g., when the piston 30 fails to advance), while some pumps 64 may exhibit an increase in end-stop switch 90 activation signal duration (e.g., when the piston 30 advances over the upstream fluid path). For example, alignment of the switch 90 on the PCB 92 with the related pump components (e.g., interlock 42, detent 40 and sleeve feature 41) may allow for some variability in what sleeve angular position releases the end-stop switch 90 and thus when the end-stop switch activation signal is generated and provided to the microcontroller 58. Additionally, high pressure in the upstream fluid path from larger insulin reservoir fill volumes may prevent the piston 30 from advancing over the upstream fluid path (e.g., resulting in a shorter pumping duration), while lower pressure in the upstream fluid path from lower insulin reservoir fill volumes may allow the piston 30 to advance over the upstream fluid path (e.g., resulting in longer or extended end-stop or limit switch activation or “trigger” duration).

With reference to FIG. 9, an example occlusion detection process comprises setting a pump measurement threshold or metric such as a switch activation duration threshold (block 96), wherein a switch activation duration below the threshold indicates normal pump operation and a switch activation duration above the threshold indicates occlusion. To set the threshold, pump measurement data can be analyzed. For example, a number of the same pumps 64 can be tested with a similar occlusion condition to collect pump measurement data related to an exhibited significant increase in the duration of a pump measurement parameter such as the end-stop switch signal voltage drop when the pump is occluded. In the case of example empirical measurements for the pump 64 in FIGS. 1, 2, 3A, 3B, 3C and 3D, switch activation durations during an occlusion measured approximately 1.5 seconds, which is commensurate with the expected amount of time for the piston 30 to translate fully through the sleeve 24. Accordingly, the occlusion detection algorithm can be configured to log end-stop switch 90 signal duration in accordance with software instructions (e.g., in the microcontroller 58) and compare the logged switch 90 activation durations against a threshold value (e.g., Th_{switch}>1.0 second(s)) to determine if an occlusion is present or not, as indicated in block 98 of FIG. 9. For example, end-stop or pump limit switch activation data can be collected and stored (e.g., via a memory device integral to the microcontroller 58 or implemented as a separate component on the PCB 92). The microcontroller 58 can be provided with an occlusion detection algorithm for processing the end-stop switch activation data to determine if an occlusion has occurred. In accordance with another illustrative embodiment, the end-stop switch activation data can be provided (e.g., wirelessly or via wireline connection) from the pump 64 to another device having an occlusion detection algorithm such as a hand-held remote controller for the pump 64 or a non-dedicated computing device (e.g., mobile phone, personal computer (PC), laptop or other portable computing device) provided with software or app comprising the occlusion sensing algorithm. The switch activation duration data for occluded pumps can be averaged or otherwise summarized or categorized, and then analyzed to determine the degree of difference between similar pump measurements for normal operating pumps and the pump measurements for the occluded pumps. The threshold (e.g., Th_{switch}) or other metric is determined to be a value or a range of values with a margin(s) above and/or below which normal pump measurements will not fall. The value, or range of values, and/or margin can be designated by a user, or automatically determined based on the pump measurement data obtained from the pump. As described above, the pump measurement data such as switch activation duration is data that is generated and monitored during the course of normal pump activity and therefore does not require an additional component that increases the complexity of the pump.

With continued reference to FIG. 9, once the pump measurement metric (e.g., switch activation duration threshold) is set, the microcontroller 58 in the medication delivery device 10 is controlled by the occlusion detection algorithm to obtain pump measurement data (e.g., switch activation duration data) for the pump 64 (block 97), and to compare the switch activation duration data to the pump measurement metric during various pump stages or cycles of operation such as for each pump cycle (block 98). When the switch activation duration data meets the pump measurement metric (e.g., is less than or equal to a Th_{switch} of 1.0 seconds), the pump is determined to be operating normally (block 100). When the switch activation duration data fails to satisfy the pump measurement metric (e.g., is greater than the occlusion detection threshold Th_{switch} of 1.0 seconds), then the pump is determined to be experiencing an occlusion condition. A counter is incremented (block 99) when a threshold Th_{switch} for normal operation is not met. With reference to block 101, when the counter reaches a selected value (e.g., the counter value of 8 corresponding to 8 pump cycles wherein a threshold Th_{switch} for normal operation is not met), then occlusion is detected. The total number of cycles during which the selected number of cycles is reached before occlusion is indicated can be designated such as 8 consecutive cycles of 8 cycles or within a designated number of cycles (e.g., 20 cycles). The microcontroller 58 can be configured by the occlusion detection algorithm to generate an optional indication of detected occlusion error (block 102), and to automatically stop operation of the pump 64 and/or the medication delivery device 10, and/or generate an optional indication to the user to cease using the medication delivery device 10 (block 104). If the counter, after being incremented per block 99, has not yet reached the selected counter value, then the pump measurement data continues to be collected per block 97. Since the occlusion detection algorithm is based on pump duration data or other pump measurement data that has already been implemented in the pump, occlusion detection is achieved by checking pump duration or other measurement data in the software against a selected threshold or metric. Accordingly, a software-only solution is provided for occlusion detected, obviating the need for any hardware changes.

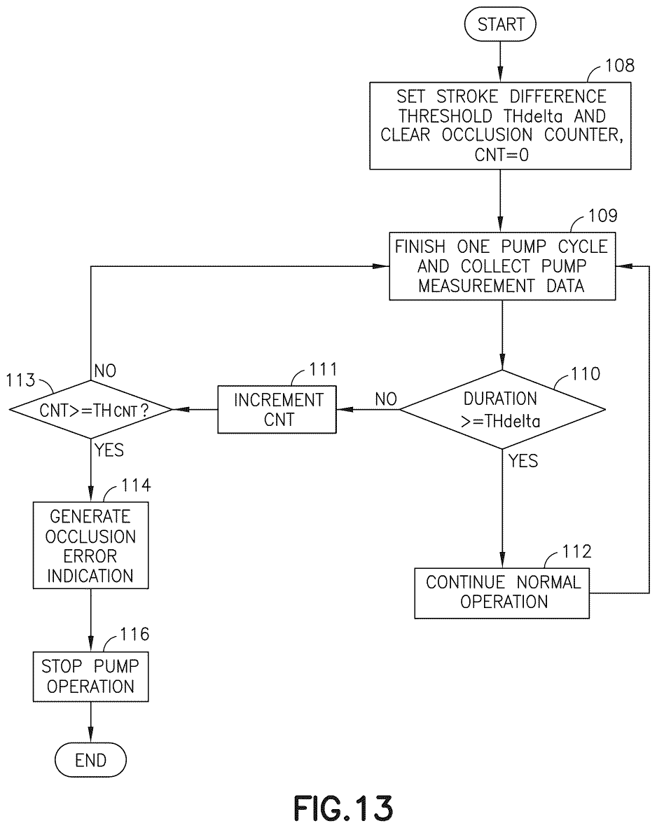

In accordance with another illustrative embodiment of the present invention, a third pump characteristic is monitored to detect an occlusion in a medication delivery device 10, as will be described below in connection with FIG. 13. For example, testing a selected pump 64 under occluded conditions revealed that, if occlusion happens when the medication delivery device 10 was new, the pump 64 tended to have short stroke duration or long end-stop duration as described above in connection with FIGS. 7 and 9, respectively. After the pump went through many cycles, however, test data indicated that it tended to leak at the joint area 49 between the manifold seal 47 and the sleeve 24, as illustrated in FIG. 3B. The reasons why there was excessive leaking after certain pump cycles was likely the combination of the wear and tear of the seal caused by the repetitive pumping motion and the high internal pressure caused by occlusion. In other words, when the pump 64 is new and the seal 47 is strong enough to tolerate the high pressure introduced by occlusion, the pump will likely exhibit a short stroke duration or long end-stop duration (e.g., prolonged limit switch activation duration) during occlusion. After some pump cycles, however, the seal is not strong enough to tolerate the high pressure introduced by occlusion, the pump 64 may leak through the weakest link of the downstream fluid path, which can be the seal 49 between the manifold 47 and the sleeve 24. Since the fluid in the pump chamber 38 is forced through the leakage path by the high internal pressure introduced by occlusion, the pump motor (not shown) needs to provide more energy to push the fluid through. As a result, the dispense stroke duration during occlusion is longer than in normal operation.

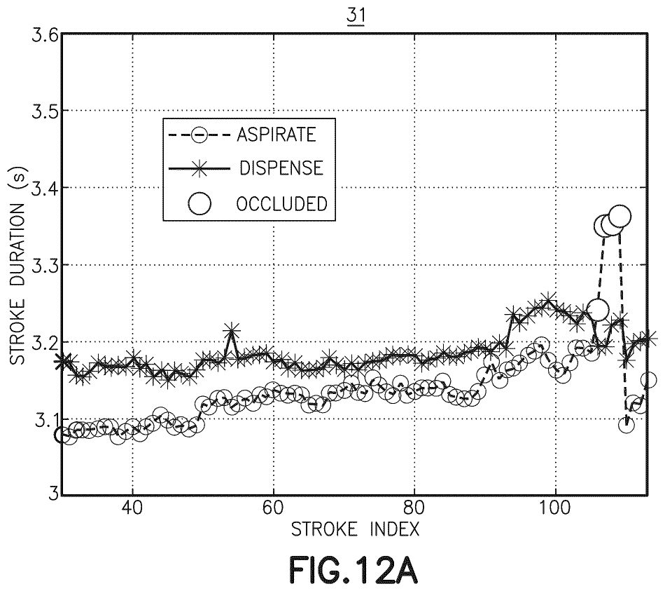

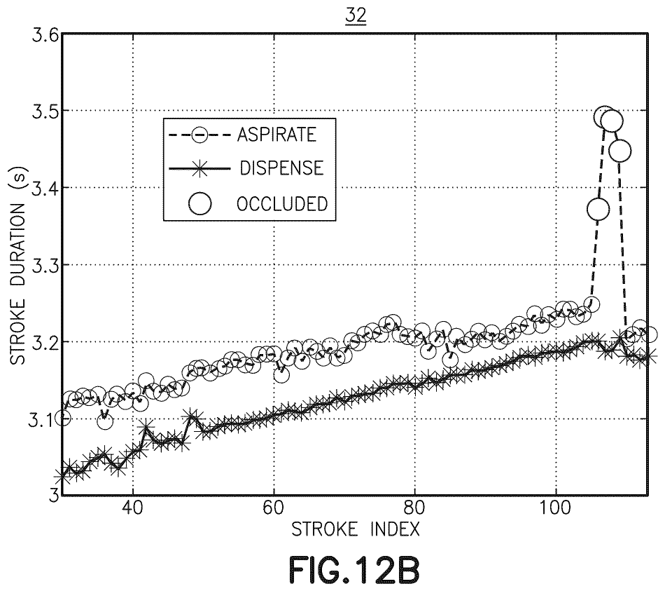

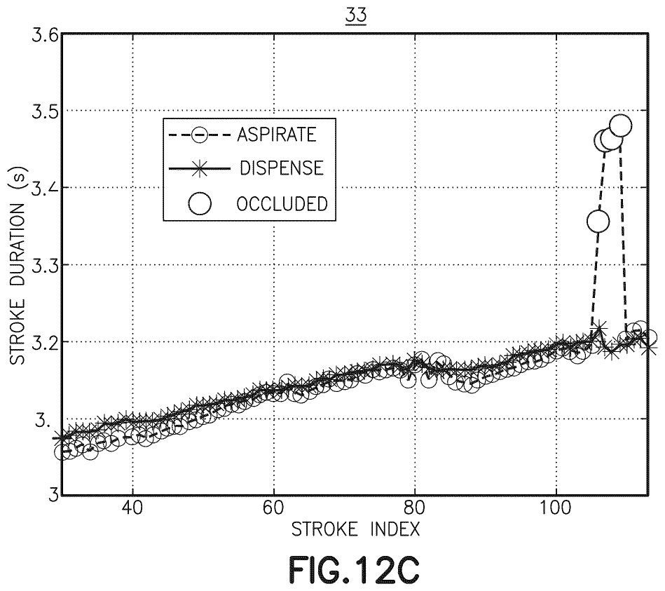

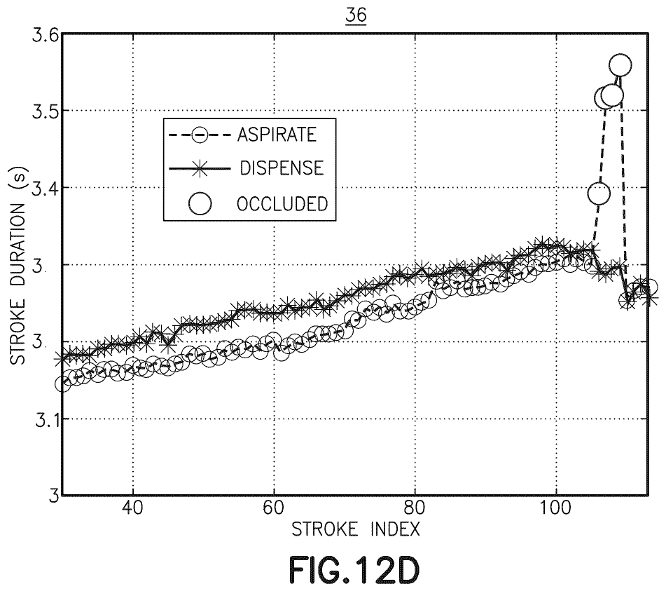

FIGS. 12A, 12B, 12C and 12D show a few examples from a bench occlusion test of a selected type of pump such as pump 64 described with respect to FIGS. 1, 2, 3A, 3B, 3C and 3D. FIGS. 12A, 12B, 12C and 12D illustrate a long dispense duration related to the leaking caused by occlusion. For four medication delivery devices 10, each plot in FIGS. 12A, 12B, 12C and 12D corresponds to one medication delivery device 10. Each medication delivery device 10 was filled, for example, with 300U fluid, and delivered 50U open, 2U clamped, and 2U open. It can be seen from these plots that, when the medication delivery devices 10 are occluded, the dispense stroke duration increases, while the aspirate stroke duration stays relatively the same. Accordingly, this pump characteristic can be used to detect leaking caused by occlusion.

In accordance with an aspect of an illustrative embodiment of the present invention, an occlusion detection algorithm as described above can employ a pump duration difference between the dispense stroke and the aspirate stroke. For example, with reference to block 108 in FIG. 13, a stroke difference threshold (Th_{delta}) can be determined as follows:

Step 1: At the end of priming, calculate the average duration difference between the aspirate stroke and the dispense stroke, defined as

$$D 0 = \frac{1}{n} {\sum }_{i = 1}^{n} [ Dispense ( i ) - Aspirate ( i ) ] ,$$

where n is number of strokes used to get the average difference. As an example, n=3 is used for the illustrative embodiment but it is to be understood that this number may vary depending on the specific pump design.

Step 2: For each pump cycle after priming, collect pump measurement data (e.g., duration difference between the aspirate stroke and the dispense stroke) for the pump 64 (block 109), and compare the duration difference data to a pump measurement metric (block 110), for example, as follows:
1) Calculate duration difference: Di=Dispense−Aspirate;
2) Subtract D0 from Di: D′i=Di−D0; and
3) Check whether D′i D′i−1, and D′i−2 are less than a given threshold (e.g., 0.13 seconds), as indicated in block 110 of FIG. 13. If yes, then normal pump operation can continue per block 112 in FIG. 13. If not, then leaking is detected and the pump may be determined to be experiencing an occlusion condition. A counter is incremented (block 111) when a threshold Th_{delta} for normal operation is not met. With reference to block 113, when the counter reaches a selected value (e.g., the counter value of 8 corresponding to 8 pump cycles wherein a threshold Th_{delta} for normal operation is not met), then occlusion is detected and an occlusion indication can be generated per block 114 and pump operation can be terminated per block 116. If the counter, after being incremented per block 111, has not yet reached the selected counter value, then the pump measurement data continues to be collected per block 109. The total number of cycles during which the selected number of cycles is reached before occlusion is indicated can be designated such as 8 consecutive cycles of 8 cycles or within a designated number of cycles (e.g., 20 cycles). Even though three consecutive dispense strokes are used in the illustrative embodiment, this number may vary depending on the variation of the pump duration over time. The duration differences D_{0, 1, . . . , x} can be averaged or otherwise summarized or categorized, and then analyzed to determine the degree of difference between the pump measurements (e.g., aspirate stroke and dispense stroke duration differences) for normal operating pumps and the pump measurements for occluded pumps, and/or with respect to a threshold or other metric Th_{delta}.

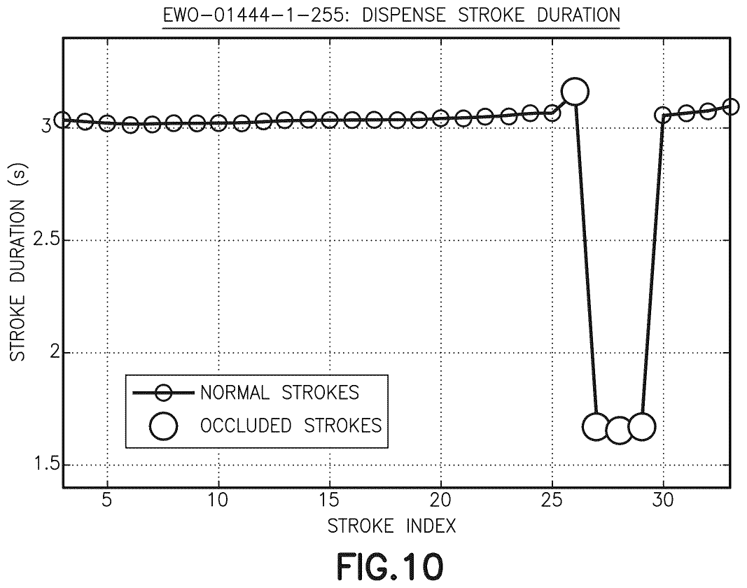

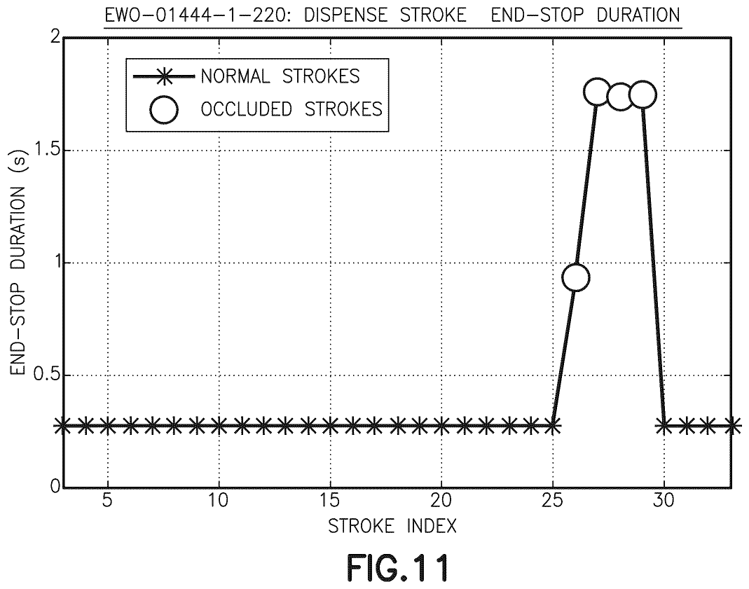

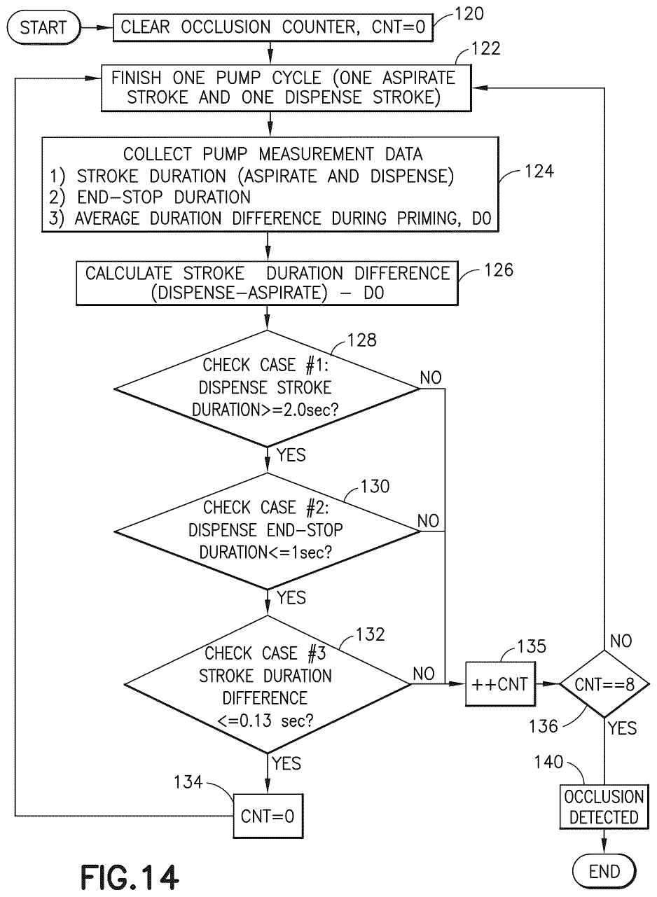

The occlusion detection algorithm can comprise the leak detection criteria described with FIG. 13, in combination with the stroke duration criteria described with FIG. 7 and/or the end-stop or limit switch activation duration criteria described with FIG. 9 in accordance with other illustrative embodiments. For example, detection using all three of the criteria or only a single criterion or subset of these three criteria can be implemented in parallel or in series using occlusion detection software provided to the microcontroller 58 or to the controller of a separate device associated with the medication delivery device 10. Additional example data for the stroke duration criteria is shown in FIG. 10, and additional example data for the switch activation duration criteria is shown in FIG. 11. With reference to FIG. 14, an example occlusion detection algorithm in accordance with an illustrative embodiment employs a combination of stroke duration criteria as described with FIG. 7, end-stop or limit switch activation duration criteria as described with FIG. 9, and leak detection criteria as described with FIG. 13. A counter for detected occlusion conditions is cleared or set to a 0 value (block 120). A pump cycle is detected (i.e., an aspirate stroke and a dispense stroke are detected using, for example, end-stop switch activation data) as indicated at block 122. Pump measurement data is collected (block 124) such as stroke duration, end-stop duration as described with reference to FIG. 9, and average duration difference (D0) between the aspirate stroke and the dispense stroke during priming. The stroke duration difference is determined (i.e., subtracting the average duration difference during priming (D0) from the duration corresponding to the dispense stroke duration less the aspirate stroke duration) (block 126). The counter is incremented (block 136) if abnormal pump operating conditions are detected such as dispense stroke duration shortening (e.g., less than a Thₛₜᵣₒₖₑ of 2 seconds) per block 128, or end-stop switch activation duration lengthening (e.g., greater than a Th_{switch} of 1 second) per block 132, or a stroke duration difference (e.g., a difference of greater than Th_{delta} of 0.13 microseconds) per block 134. When the counter reaches a selected value (e.g., the counter value of 8 corresponding to 8 pump cycles wherein a threshold for normal operation is not met) per block 138, then occlusion is detected per block 140 and an occlusion indication can be generated and/or pump operation can be terminated, for example. If none of these occlusion conditions are met, the counter remains cleared (e.g., 0 value) per block 134, and the next pump cycle is detected and related pump timing or measurement data is collected per block 122.

With regard to block 132 in FIG. 14, the threshold Th_{delta} can be an adaptive threshold rather than a fixed threshold. For example, for each dispense stroke, if the stroke duration difference in block 126 is greater than or equal to an adaptive threshold for Th_{delta}, then the counter is incremented (block 136). The adaptive threshold for Th_{delta} is a moving average of “(Dispense duration−Aspirate duration)−D0” plus a fixed offset. The fixed offset can be predefined in the occlusion detection algorithm (e.g. 0.09 sec). The window size of the moving average can be 10 pump cycles and also predefined in the occlusion detection algorithm implemented in accordance with software instructions by the microcontroller 58, for example. Accordingly, is averaging for a given pump cycle, the adaptive threshold for Th_{delta} is the average of 10 previous pump cycles plus the offset. The averaging can start at the first full priming cycle of the pump. If there is less than 10 pump cycles for a given pump cycle, the moving average can be based pn an average of the available previous pump cycles. A pump cycle can be excluded from the moving average calculation if any of the following conditions are met: the occlusion counter is incremented in a previous step (e.g. blocks 128 and 130 in FIG. 14; the current pump cycle is determined to be above the adaptive threshold for Th_{delta}; or the previous pump cycle is above the adaptive threshold for Th_{delta} while the current pump cycle is below the adaptive threshold for Th_{delta}.

By way of an example, the leak detection criteria described with the occlusion detection algorithm in connection with FIG. 13 above was applied to the bench occlusion data collected from 280 medication delivery devices 10 in combination with the short stroke duration algorithm (e.g., described above with reference to blocks 80 and 82 in FIG. 7) and the long end-stop duration algorithm (e.g., described above with reference to blocks 96 and 98 in FIG. 9). Table 1 shows the comparison between without and with the leak detection algorithm described with reference to blocks 108 and 110 in FIG. 13. It can be seen that the leak detection algorithm (e.g., blocks 108 and 110 in FIG. 13 and incorporated as block 132 in FIG. 14) significantly improved the correct detection rate of occlusion by the occlusion detection algorithm in accordance with illustrative embodiments of the present invention. However, it increases the false positive rate slightly.

TABLE 1
Occlusion Detection w/ and w/o Leak Detection

| | | | | # False | |
|---|---|---|---|---|---|
| | | | # False | Positive | Correct |
| | Total # | # | Negative | (False | Detection |
| | Samples | Detected | (Miss) | Alarm) | Rate |
| w/o Leak | 280 | 89 | 181 | 0 | 32% |
| Detection | | | | | |
| w/ Leak | 280 | 270 | 5 | 5 | 96% |
| Detection | | | | | |

Out of the 280 medication delivery devices 10, there were 120 medication delivery devices 10 that delivered a 10U bolus before clamping. The manifold seals 49 in these medication delivery devices 10 were minimally used. Table 2 shows the comparison between without and with the leak detection algorithm for this medication delivery devices 10 group. It can be seen from Table 2 that if the manifold seals 49 are minimally used, the occlusion detection rate is quite high, 88%, even without the leak detection algorithm added to the occlusion detection algorithm employing analysis of stroke duration measurements and/or long end-stop duration pump measurements. These results are consistent with the fact that the leak is mostly caused by the wear and tear of the manifold seal after repetitive pumping motions.

TABLE 2
Occlusion Detection with and without Leak
Detection for a subgroup of medication delivery devices
10 (10 U bolus before clamping)

| | | | | # False | |
|---|---|---|---|---|---|
| | | | # False | Positive | Correct |
| | Total # | # | Negative | (False | Detection |
| | Samples | Detected | (Miss) | Alarm) | Rate |
| w/o Leak | 120 | 106 | 14 | 0 | 88% |
| Detection | | | | | |
| w/ Leak | 120 | 120 | 0 | 0 | 100% |
| Detection | | | | | |

Accordingly, a leak detection criteria can be implemented in the occlusion detection algorithm. Since this algorithm only needs pump duration information to analyze leak detection criteria, there is no hardware change required. The occlusion detection algorithm employing leak detection criteria is improved when implemented in tandem with the stroke duration criteria and/or the end-stop switch activation duration criteria in order to more fully capture all significant pump behaviors during an occlusion.

In accordance with yet another illustrative embodiment, pump motor current is used to detect occlusion. Under the normal working condition, the medication delivery device 10 described above aspirates from the reservoir 70, which is at the upstream of the fluid path, and dispenses to patient body, which is at the end of the downstream fluid path. During the aspirate stroke, the piston opens the pump chamber, which allows the fluid from the reservoir to fill the chamber. During the dispense stroke, the piston closes the pump chamber, which pushes the fluid to the downstream. FIGS. 3A and 3B depict an example piston and the pump chamber.

When the medication delivery device 10 is occluded, the piston cannot empty the fluid inside the pump chamber to the downstream. As a result, the pump May 1) hold the fluid inside the pump chamber, 2) pump the fluid back to the reservoir, or 3) the fluid may be forced to leak through the manifold seal of the pump. Since it takes more energy to pump the fluid to any of these three pathways, the motor current is higher during the dispense stroke when occlusion happens. Therefore, motor current can be used to detect occlusion.

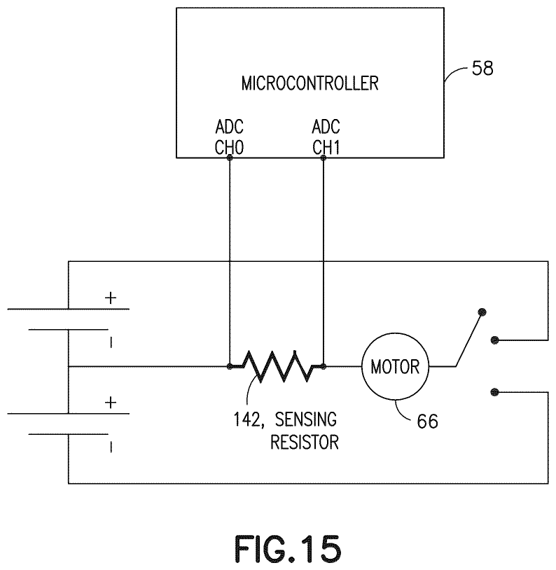

FIG. 15 shows an example apparatus for motor current sensing. A sensing resistor 142 is added to the PCB 92 to enable motor current measurement. The voltage drop on the sensing resistor 142 is provided into the analog-to-digital converter (ADC) of the microcontroller 58. The occlusion condition is then calculated by the microcontroller 58, and an occlusion event is reported by the microcontroller 58 when, for example, a designated occlusion signature is detected. Other components can be used for current sensing to facilitate pump motor current measurement. For example, for a pulse width modulation (PWM) drive motor used as a pump actuator 66, motor current information can be extrapolated from PWM data.

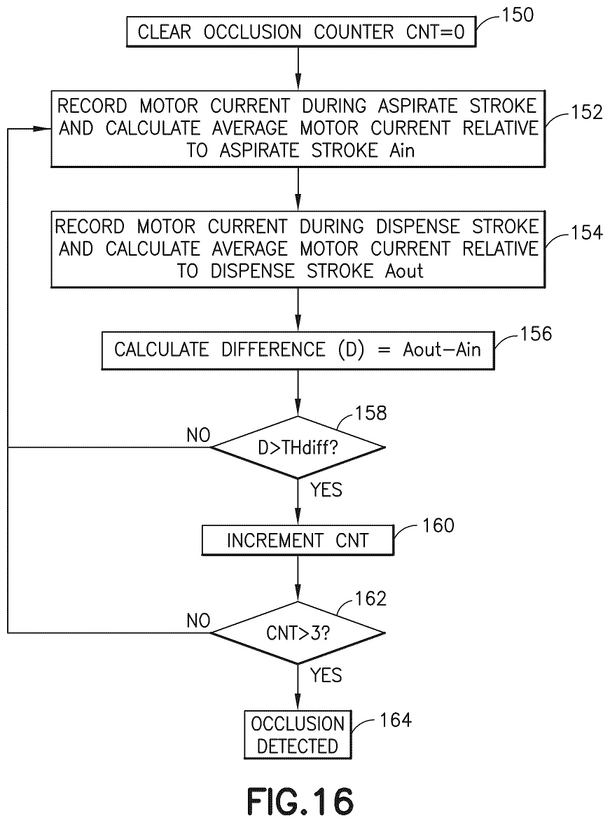

An illustrative occlusion detection algorithm for each pump cycle is described below with reference to FIG. 16. A counter for detected occlusion conditions is cleared or set to a 0 value (block 150). Motor current is determined during an aspirate stroke of a pump cycle (block 152) For example, at the start of an aspirate stroke, motor current is recorded via the microcontroller 58 during the aspirate stroke. Using xᵢₙ(t), where t is the time referencing to the beginning of this stroke, at the conclusion of the aspirate stroke (e.g., when an end-stop signal is detected), the microcontroller 58 can be programmed to determine average motor current Aᵢₙ between 1 seconds (sec) and 2.5 sec relative to the start of the motor current as follows:

$${A}_{in} = mean [ {x}_{in} ( t ) , 1 sec < t < 2.5 sec ] .$$

It is to be understood that other methods of determining motor current during a pump cycle or aspirate stroke or dispense stroke can be used.

Motor current is also determined during a dispense stroke of a pump cycle (block 154). For example, at the start of a dispense stroke, the microcontroller 58 records motor current during the dispense stroke. Using Xₒᵤₜ(t), where t is the time referencing to the beginning of this stroke, at the conclusion of the dispense stroke (e.g., when a corresponding end-stop signal is detected), the microcontroller 58 can be programmed to determine average motor current Aₒᵤₜ between 1 sec and 2.5 sec relative to the start of the motor current, as follows:

$${A}_{out} = mean [ {x}_{out} ( t ) , 1 sec < t < 2.5 sec ] .$$

With reference to block 156 of FIG. 16, the microcontroller 58 is configured to calculate the motor current difference (D) between the aspirate stroke and the dispense stroke denote as D, where

$$D = {A}_{out} - {A}_{in} .$$

If the difference (D) is larger than a designated threshold Th_{iDiff} (block 158), a counter is incremented (block 160). With reference to block 162, when the counter reaches a selected value (e.g., the counter value of 3 corresponding to 3 pump cycles wherein a threshold Th_{iDiff} for normal operation is not met), then occlusion is detected and an occlusion indication can be generated per block 164 and pump operation can be terminated. It is to be understood that the counter value can be another value than 3 for designating a different number of cycles over which the pump current exceeds a threshold before occlusion is indicated as detected. If the counter, after being incremented per block 160, has not yet reached the selected counter value (block 162), then the pump measurement data (e.g., motor current) continues to be collected per block 152. Thus, if the latest pump cycle and a few consecutive previous pump cycles have D values larger than a given threshold, occlusion is indicated; otherwise, normal operation of the pump is continued.

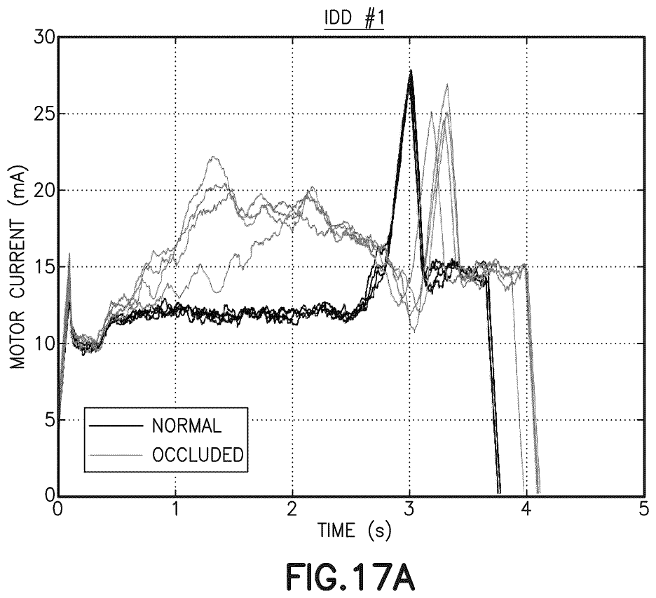

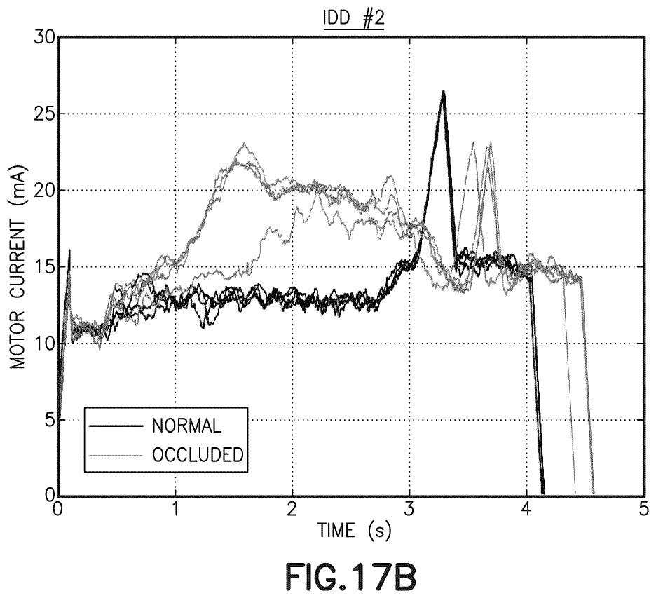

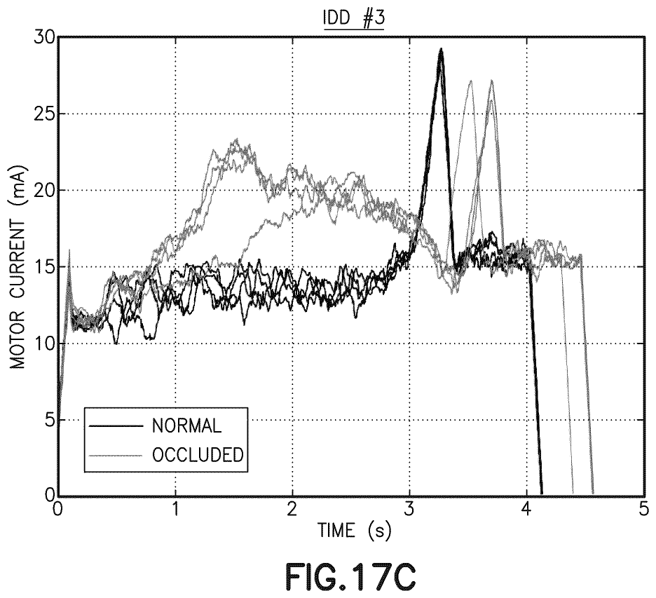

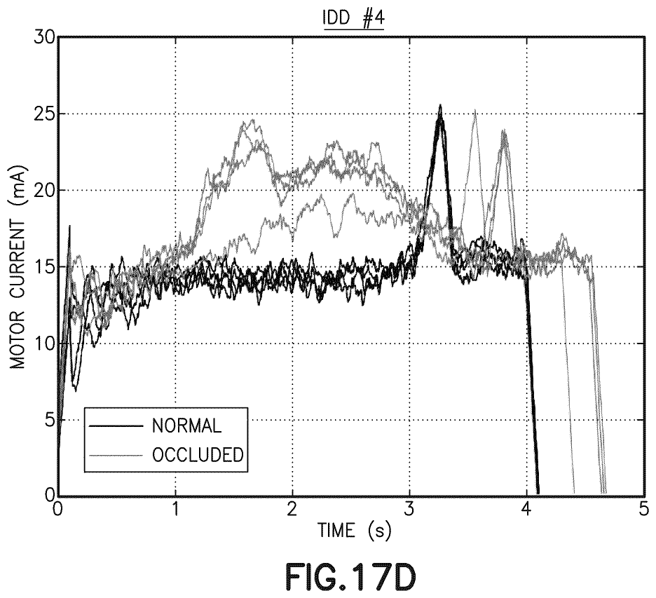

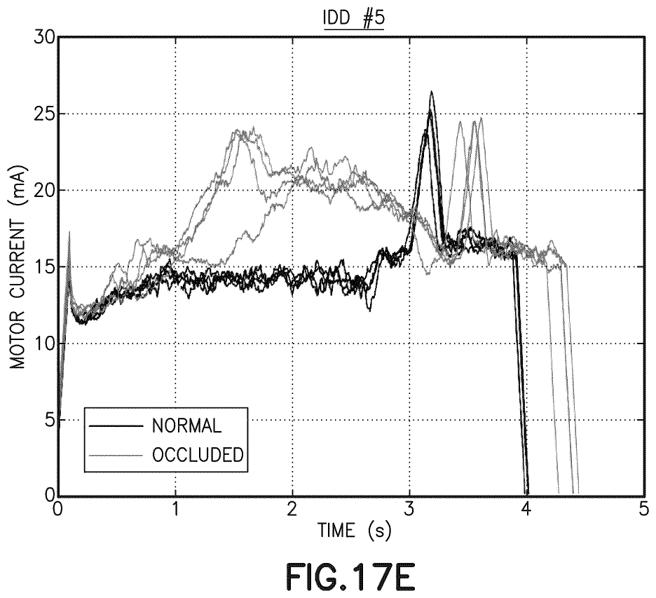

FIGS. 17A through 17E each show motor current during a dispense stroke before and after occlusion measured from five respective example delivery devices 10. FIGS. 17A through 17E depict a clear distinction of motor current between the normal and the occluded pump strokes, which facilitates use of an occlusion detection algorithm based on motor current such as the algorithm described above in connection with FIG. 16.

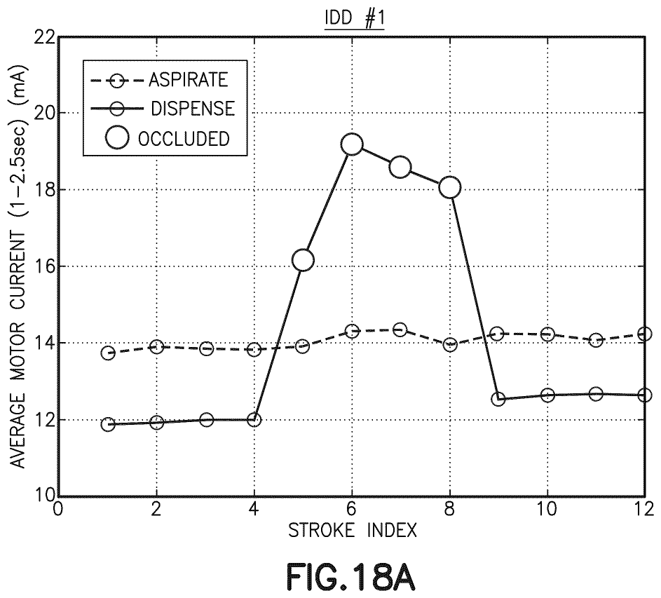

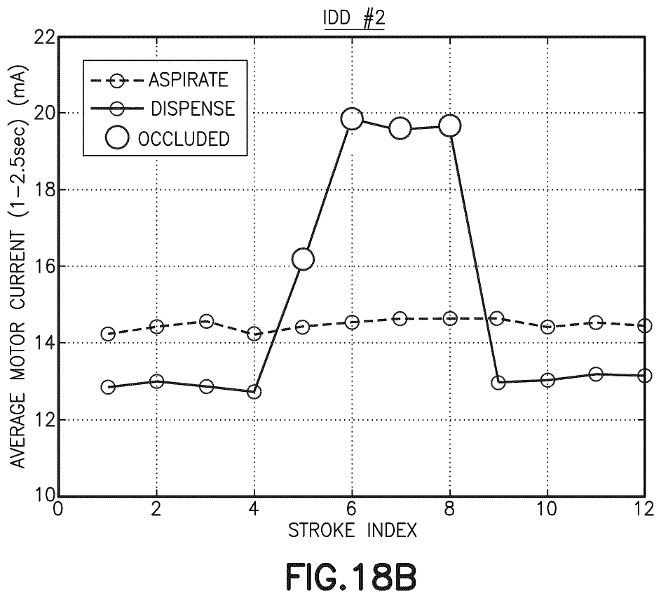

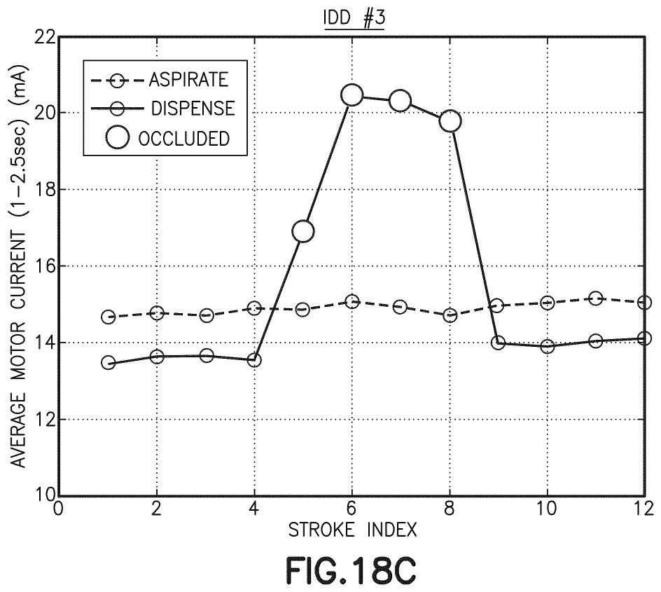

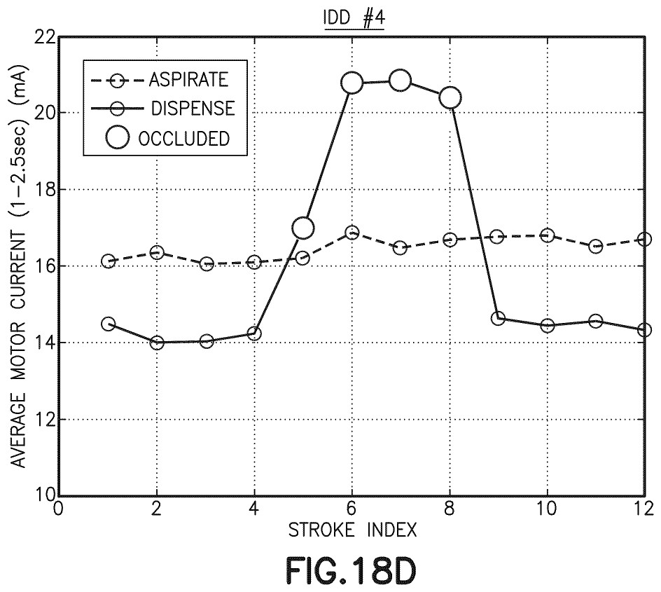

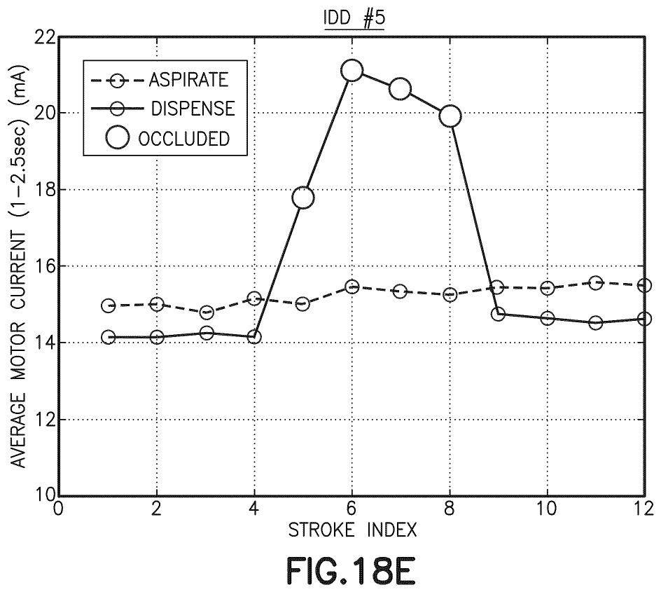

FIGS. 18A through 18E each show the average motor current from 1 second to 2.5 seconds (i.e., measured over a duration of 1-2.5 seconds after a stroke commences where t=0 is the start of the stroke). Again, there is clear distinction between occluded strokes and normal strokes illustrated in FIGS. 18A through 18E. Accordingly, occlusion can be detected by applying a threshold Th_{iDiff} to the averaged motor current.

With continued reference to FIGS. 16, FIGS. 17A through 17E, and FIGS. 18A through 18E, an alternative approach can be to only rely on average motor current for the dispense stroke and not also the aspirate stroke, in which case calculation of average motor current for aspirate strokes and D=Aout−Ain would not be needed. For example, such an alternative algorithm can comprise the following operations: motor current is determined during a dispense stroke of a pump cycle. For example, at the start of a dispense stroke, the microcontroller 58 records motor current during the dispense stroke. Using xₒᵤₜ(t), where t is the time referencing to the beginning of this stroke, at the conclusion of the dispense stroke (e.g., when a corresponding end-stop signal is detected), the microcontroller 58 can be programmed to determine average motor current Aₒᵤₜ between 1 sec and 2.5 sec relative to the start of the motor current, as described above in connection with FIG. 16. If the average motor current Aₒᵤₜ is larger than a designated threshold Th_{Aout}, a counter is incremented. When the counter reaches a selected value (e.g., the counter value of 3 corresponding to 3 pump cycles wherein the threshold Th_{Aout} for normal operation is not met), then occlusion is detected and an occlusion indication can be generated and pump operation can be terminated. It is to be understood that the counter value can be another value than 3 for designating a different number of cycles over which the pump current exceeds a threshold before occlusion is indicated as detected. If the counter, after being incremented, has not yet reached the selected counter value, then the pump measurement data (e.g., motor current) continues to be collected. Using motor current data from both the aspirate stroke and the dispense stroke as described above with FIG. 16; however, is likely to be more robust in terms of sensitivity and accuracy of occlusion detection using motor current, for example.

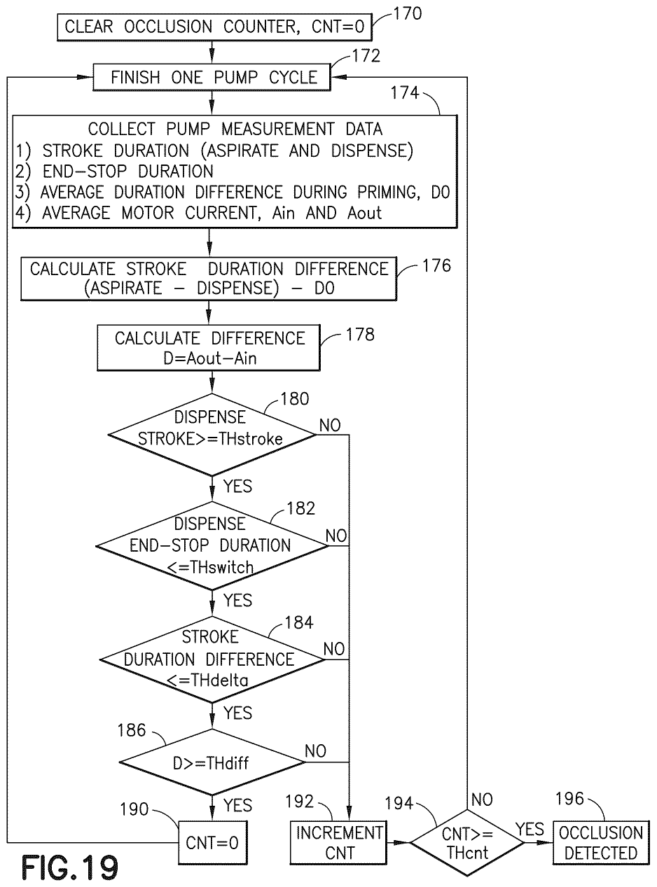

With reference to FIG. 19, the occlusion detection algorithm can comprise the motor current criteria described with FIG. 16, in combination other criteria used to detect an occlusion. For example, an example occlusion detection algorithm in accordance with an illustrative embodiment in FIG. 19 employs a combination of motor current criteria described with FIG. 16 with stroke duration criteria as described with FIG. 7, end-stop or limit switch activation duration criteria as described with FIG. 9, and leak detection criteria as described with FIG. 13. A counter for detected occlusion conditions is cleared or set to a 0 value (block 170). A pump cycle is detected (i.e., an aspirate stroke and a dispense stroke are detected using, for example, end-stop switch activation data) as indicated at block 172. Pump measurement data is collected (block 174) such as stroke duration, end-stop duration as described with reference to FIG. 9, and average duration difference between the aspirate stroke and the dispense stroke during priming, and average motor current during each of the aspirate stroke and the dispense stroke, for example. The stroke duration difference is determined (i.e., subtracting the average duration difference during priming from the duration corresponding to the dispense stroke duration less the aspirate stroke duration (block 176). The difference (D) in the average motor current for the dispense stroke as compared with the aspirate stroke is also calculated (block 178). The counter is incremented (block 192) if abnormal pump operating conditions are detected such as dispense stroke duration shortening (e.g., less than a Thₛₜᵣₒₖₑ of 2 seconds) per block 180, or end-stop switch activation duration lengthening (e.g., greater than a Th_{switch} of 1 second) per block 182, or a stroke duration difference (e.g., a difference of greater than Th_{delta} of 0.13 microseconds) per block 184, or a difference in average motor current as between dispense and aspirate strokes that is larger than a designated threshold Th_{Diff}. per block 186. When the counter reaches a selected value (e.g., the counter value of 8 corresponding to 8 pump cycles wherein a threshold for normal operation is not met) per block 194, then occlusion is detected per block 196 and an occlusion indication can be generated and/or pump operation can be terminated, for example. If none of these occlusion conditions are met, the counter remains cleared (e.g., 0 value) per block 190, and the next pump cycle is detected and related pump timing or measurement data is collected per block 172. It is to be understood that one or more of the blocks 180, 182, 184 and 186 and their corresponding pump measurement data collection or calculation can be omitted to achieve an alternative illustrative algorithm that employs the remaining ones of the blocks 180, 182, 184 and 186.

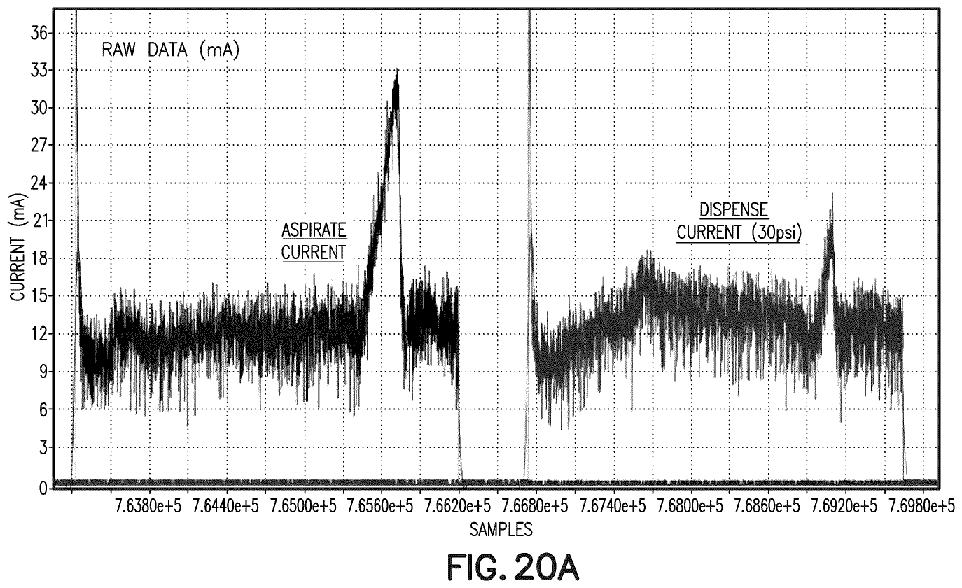

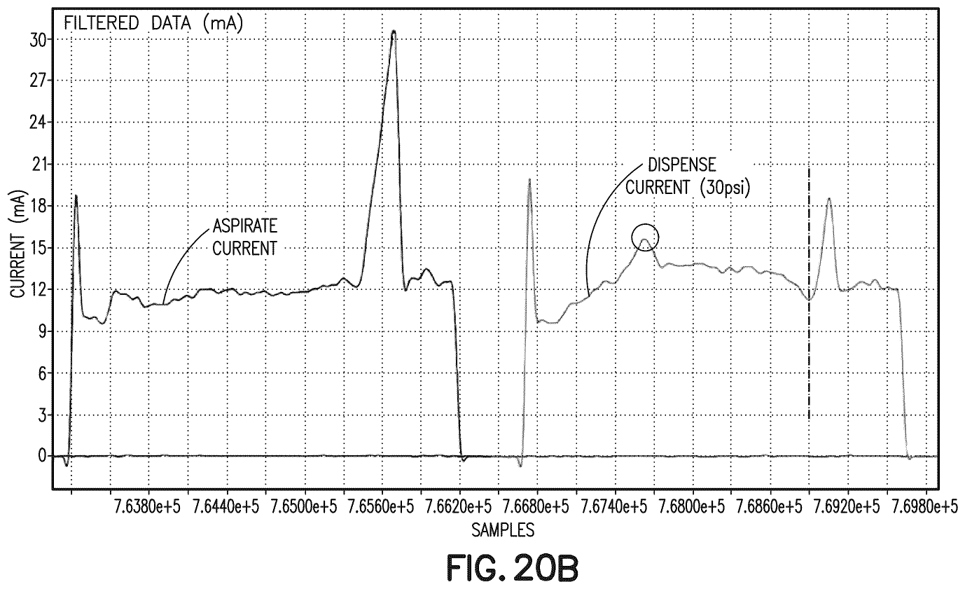

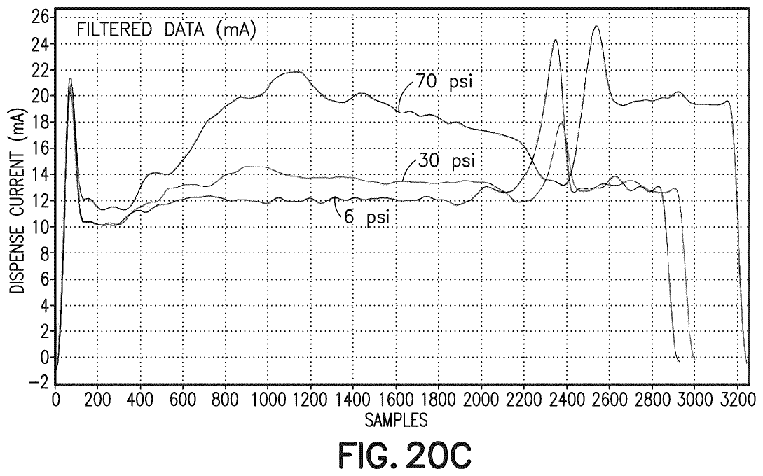

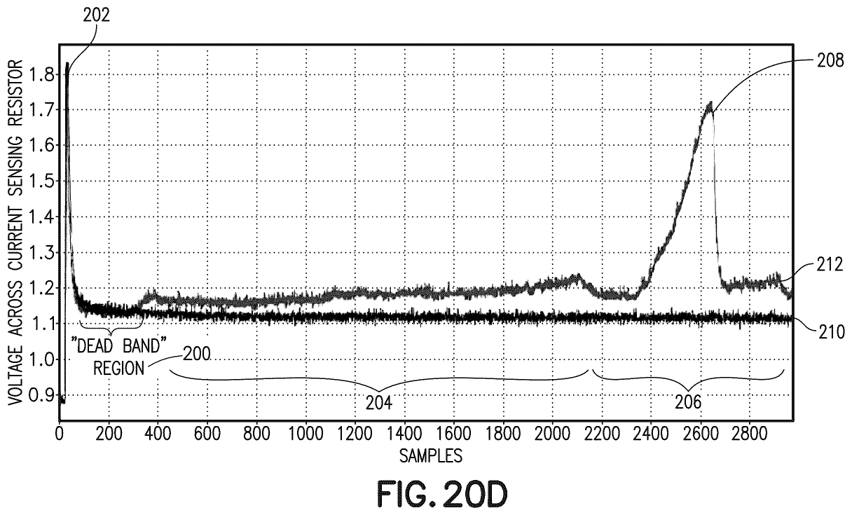

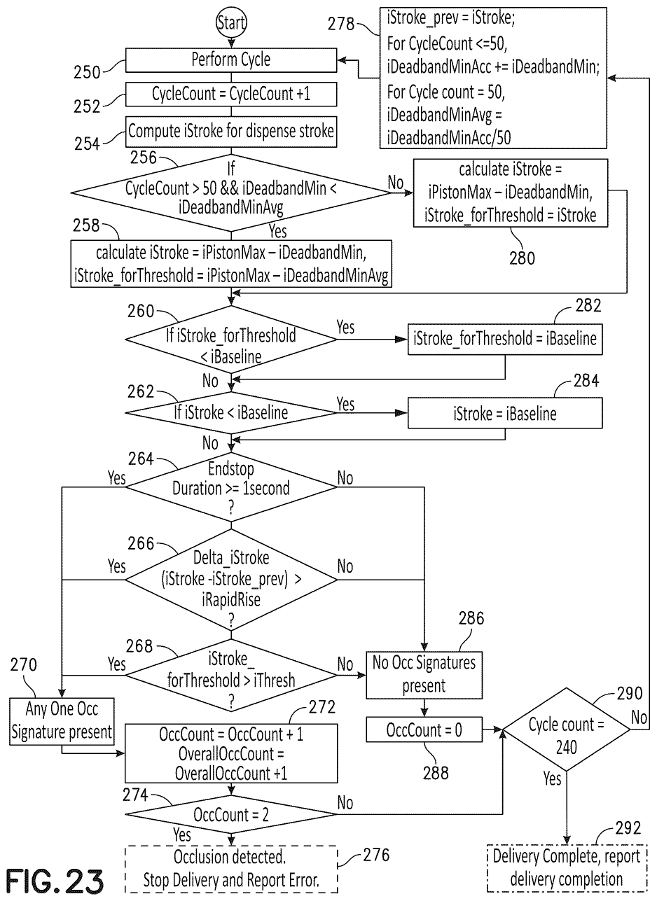

Alternatively, the occlusion sensing using motor current described in connection with FIG. 16, and block 186 in FIG. 19, can be replaced with another occlusion detection algorithm that uses motor current as described below in connection with FIGS. 20 through 23. An overview will now be described followed by a description of this alternative occlusion detection algorithm shown in FIG. 23. A fluid delivery device such as a wearable patch pump 10 has a motor 66 that rotates and moves a gearbox 33 and pump 20, 64 together. The patch pump 10 can be configured to measure a voltage (e.g., as described above in connection with FIG. 15) and then use the measured voltage to calculate motor current, which is related to pump 20, 64 torque and therefore to downstream pressure in the patch pump 10. For example, the patch pump 10 can require a high current and therefore a high torque to deliver fluid to a patient due to a blockage in the downstream fluid path, or high in vivo backpressures, or other issue with the patch pump components that can cause an increase in pump torque and possibly lead the pump 20, 64 to stall. The occlusion detection algorithm searches the motor 66's current data looking for local extremes in two time-based windows. The first window is used to identify when the motor 66 first moves the gearbox 33 in isolation without the pump 20, which is hereinafter referred to as the “dead band” region and current measured therein is basically the current drawn “iDeadband” to move the gearbox 33 and motor 66 alone, as described more below. The second window is a region wherein measured current therein is used to identify the maximum current drawn to move the plunger or piston 30 and the gearbox 33 and the motor 66 and is referred to as “iPistonMax.” A value iStroke is determined as iPistonMax−iDeadband and corresponds to motor current drawn to move the piston 30 alone. iStroke can be related to downstream pressure. Occlusion alarms can be generated based on various conditions involving changes in iStroke described below. As stated above, the patch pump can detect occlusion using motor current in combination with some time based conditions such as one or more of those described above in connection with FIGS. 7, 9 and 13. For example, when the pump 20 rotates from an upstream to downstream flow position or vice versa, a feature 41 on the pump 20 can actuate a switch 90 on the printed circuit board 92 as described above in connection with FIG. 3C. If the time this switch 90 is engaged on a dispense stroke (e.g., end-stop switch 90 duration) exceeds a defined time limit, an occlusion alarm is generated as described above in connection with FIG. 9. In addition, if a pump cycle duration (e.g., time to complete aspirate and dispense strokes) exceeds a defined time limit, a pump timeout alarm can be generated that is not the same as an occlusion alarm, but can forms a part of an occlusion detection algorithm. The pump cycle duration is not taken from motor current, but rather relies on the switch 90 on the printed circuit board 92 to be activated by the pump 20 twice (i.e., once upon the aspirate stroke and once upon the dispense stroke of a pump cycle) to know the pump cycle duration.

As stated above, current sensing is a method of detecting occlusions in the fluid path of a fluid delivery device such as an infusion pump because an occlusion causes a decrease in flow of medicament or other fluid to be delivered to a patient, which causes increased pressure. Increased pressure causes increased torque demand on the pump motor, and increased torque demand by the motor draws more current. Other motor parameters besides motor current such as motor voltage and encoded count can be also used to detect increased pressure. Many other design factors, however, can affect current demand by motors, as well as other motor parameters, including, but not limited to, gearbox efficiency, pump seals and their wear over time, motor efficiency, and motor magnet angle. In addition, there are environmental factors like ambient pressure and temperature that can affect motor current demand. These factors can negatively impact the accuracy of using a measured pump motor parameter such as motor current to detect occlusion.

Example embodiments in the present disclosure provide a technical solution to the above described problems. Because of the afore-mentioned design and environmental factors that impact demand on a pump motor, it is extremely important to adjust or calibrate a pump motor signal used to detect occlusions in a fluid delivery device such as an infusion pump so that only changes to the motor signal that are due to changes in pressure are measured and used for occlusion detection and not factors unrelated to pressure such as changes over time in the battery, motor and gearbox from wear, ambient temperature changes, differences in pump performance during aspirate versus dispense operations, and so on. An ideal normalization compensates for everything but pressure, and the example embodiments and technical solution provided herein are advantageously close to ideal normalization.

The technical solution and example embodiments provided in the present disclosure employ dead band normalization; that is, adjusting or normalizing measured data related to a fluid movement operation controlled by a drive mechanism in a fluid delivery device to data obtained during a dead portion of that operation when the drive mechanism is not moving fluid). The technical solution and example embodiments provided in the present disclosure advantageously employ dead band normalization to improve accuracy of detecting occlusion or other condition by using the normalized measured data. The measured data can be, for example, motor current during a fluid dispense or aspirate operation. As used herein, “loaded” measured data refers to the measured data obtained during a fluid movement operation when the drive mechanism is moving fluid, and “unloaded” measured data refers to the measured data obtained during a dead portion of the fluid movement operation when fluid is not being moved by the drive mechanism. Dead band normalization is understood to mean that loaded measured data is adjusted or normalized to the unloaded measured data during a particular fluid movement operation in a fluid delivery device. “Dead band normalizing” and “dead band normalization” as used herein are advantageous because they remove unwanted signal noise components and/or the effects of undesirable variants related to the drive mechanism in a fluid delivery device (e.g., a pump motor in a medication infusion device) from measured data. Removal of unwanted signal noise or undesirable impacts of noise factors (e.g., motor design or environmental factors) from measured r data can involve, for example, subtracting a averaged unloaded measured data from loaded measured data obtained while a pump motor is operated to move fluid. Dead band normalization can also involve other mathematical adjustment or calibration operations besides subtraction to normalize measured loaded pump motor data to measured unloaded pump motor data such as dividing an averaged loaded measured signal by an averaged unloaded measured signal.

While there may be different options for normalizing measured data such as pump motor current in a fluid delivery device, dead band normalizing to a dead band region of the measured data, as illustrated by example embodiments described below, realizes significant advantages in terms of accuracy of detecting a selected delivery device condition based on measured data. For example, one way to normalize pump motor data might be to normalize the measured data obtained during a dispense operation of the pump to the data obtained during a previous aspirate operation because the aspirate operation is not affected by downstream pressure. However, the aspirate operation is affected by different factors than a dispense operation such as upstream pressure, reservoir fill volume, and other noise factors which do not affect aspirate and dispense operations evenly. For example, normalizing measured pump motor data to an aspirate operation of the pump effectively doubles the noise in the measured signal and introduces noise factors not present with dead band normalization as provided by the technical solution described in the present disclosure.

Different factors impacting motor current are, for example, motor winding resistance, applied voltage (e.g., which changes with battery age), and motor speed. In addition, current during a dispense or aspirate operation can be impacted by gear train losses, motor friction losses, and drive mechanism (e.g., piston) friction losses. An advantage of the technical solution described herein is that a desired factor (i.e., pressure during an aspirate operation PA or a dispense operation PD) can be obtained by removing all of the other constants and factors related to friction losses and battery changes using dead band normalization in accordance with technical solution and example embodiments thereof described herein.

See, for example, FIGS. 20A and 20B that depict, respectively, raw and filtered pump measurement data (e.g., motor current) from an example fluid delivery device during aspirate and dispense operations. FIG. 20C depicts filtered pump measurement data from an example fluid delivery device indicating motor current during dispensing and variance at different pressures; however, all of the depicted measured current signals share a relatively similar waveform shape comprising a first spike 202 corresponding to motor start up, a portion 204 of the shape corresponding to piston movement, followed by a portion 206 corresponding to a valve state change and an interlock torque spike 208 related to a rotational metering-type pump described below herein connection with FIGS. 1 through 4 and 15.

Dead band normalization of a measured pump motor signal such as a motor current signal during a dispense operation involves obtaining the current signal when the motor gearbox is turning but is not engaging the pump. See, for example, FIG. 20D wherein two superimposed current signal waveforms are shown. One of the waveforms 212 is obtained during a dispense operation of the rotational metering-type pump, for example, and comprises a motor start-up spike 202, a piston movement portion 204, a valve state change 206 and interlock torque spike 208). The other waveform 210 is obtained during motor operation without the pump (e.g., the motor is disengaged from the pump drive mechanism). Both of these waveforms have a similar portion 200 corresponding to a dead band region that can be identified, and the data obtained therein during a dispense operation can be used for dead band normalization of measured pump motor data obtained during motor and pump operations to more accurately detect occlusion conditions by reducing noise with removal of undesirable variability of factors impacting the motor. Dead band normalizing to this part of the signal is good because: (1) this part of the measured pump motor parameter signal is nearer in time to the portion of the measured signal that is of interest (e.g., measure current during piston movement to determine pressure changes that may indicate occlusion) which inherently reduces noise because noise factors change with time; and (2) variations in the battery, motor, and gearbox are dead band normalized out of the analyzed signal because it is essentially the same as the signal from just those components. A noise factor(s) is understood to mean a factor(s) that introduces variability either internally or externally to the fluid delivery device system, or subsystem, or part thereof such as temperature, humidity, part-to-part variation, part wear, etc.

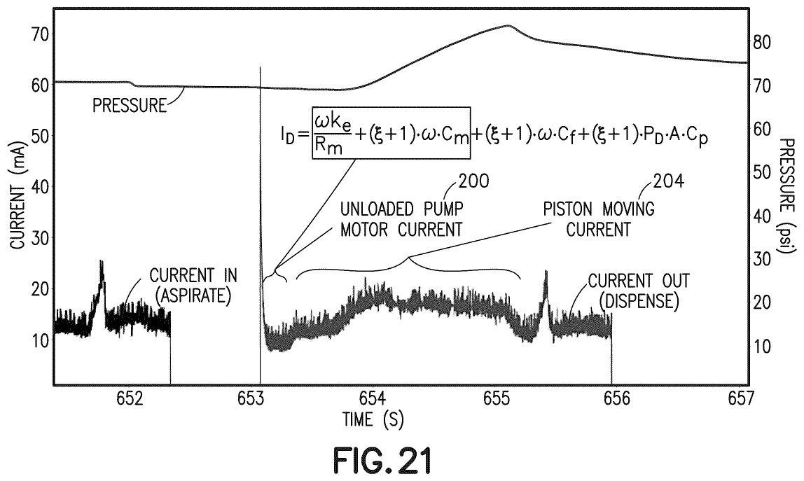

It is to be understood that the dead band region 100, or timing during a dispense or aspirate operation for obtaining dead band normalization data, can differ depending on the type of pump and pump drive mechanism. For example, a syringe-type pump as described in connection with FIGS. 4-11 in the afore-mentioned commonly owned International PCT patent publication no. WO 2022/132555 can be operated, at any time during a dispense operation, to temporarily disengage the pump drive mechanism (e.g., reverse its direction so that it is not pushing a plunger in a syringe-type reservoir to dispense the fluid) to obtain unloaded measured data while fluid is not being moved during the dispense operation. This unloaded measured data, in turn, is used to dead band normalize loaded measured pump motor data obtained during pump engagement that results in dispensing of fluid. For a syringe-type pump that is filled manually (e.g., by a supply syringe coupled to an inlet port of the syringe-type reservoir of the pump), the motor can be controlled to perform a controlled aspirate operation wherein a controlled retraction of the pump piston draws back a plunger in the syringe-type reservoir to controllably intake more fluid from the supply reservoir into the fluid chamber of the syringe-type reservoir to obtain loaded measured data while fluid is being moved. This loaded measured data during a controlled aspirate movement can be dead band normalized to unloaded measured data obtained during manual filling when the drive mechanism is not being operated to move fluid. Alternatively, for a rotational metering-type pump as explained below in connection with FIGS. 1 through 4 and 15, the dead band region 200 can occur at the beginning of each aspirate stroke and each dispense stroke as described in connection with FIGS. 20B, 20D and 21.

To optimize use of dead band normalization in accordance with the technical solution provided herein, the fluid delivery device has an interface as close to its fluid driving interface as possible that can move without moving the fluid. As described in connection with a syringe-type pump (i.e., in the afore-mentioned WO 2022/132555), this interface can be a plunger driver component such as a pusher configured on the end of a drive mechanism that can abut a reservoir plunger after the reservoir is filled and be controlled to push the plunger towards the distal end of the reservoir to dispense fluid therefrom. For dead band normalization, the pusher can be driven backward and thereby disengaged from the plunger. During a dispense operation, the pusher can be driven forward again to reengage the plunger. By being driven backward, the controller operating in accordance with a dead band normalization algorithm has essentially of all of the same effects of the pump motor driving the pusher forward (e.g., to dispense) except for plunger friction and force that results from pressure. In the case of a syringe-type pump configured without a pusher (e.g., its drive mechanism is connected directly to its plunger), the interface can be the plunger being retracted during dispensing by a nominal amount to obtain unloaded measured data without causing unwanted retrograde fluid flow. As described in connection with FIG. 3D in the case of a rotational metering-type pump, this interface can be a gap between an output gear of the gearbox 33 and a piston tab 31 such that, when the gearbox changes direction, there is a period of time when the piston 30 is not engaged at all (e.g., dead band region 200 in FIG. 21) and therefore facilitates dead band normalization to work. Thus, the technical solution and example embodiments thereof described in the present disclosure advantageously employs a loose fitting drive train feature to obtain and use unloaded data from a dead band region of a fluid movement operation and therefore is very different from existing methods of reducing noise and efforts to improve accuracy of pump motor data readings.

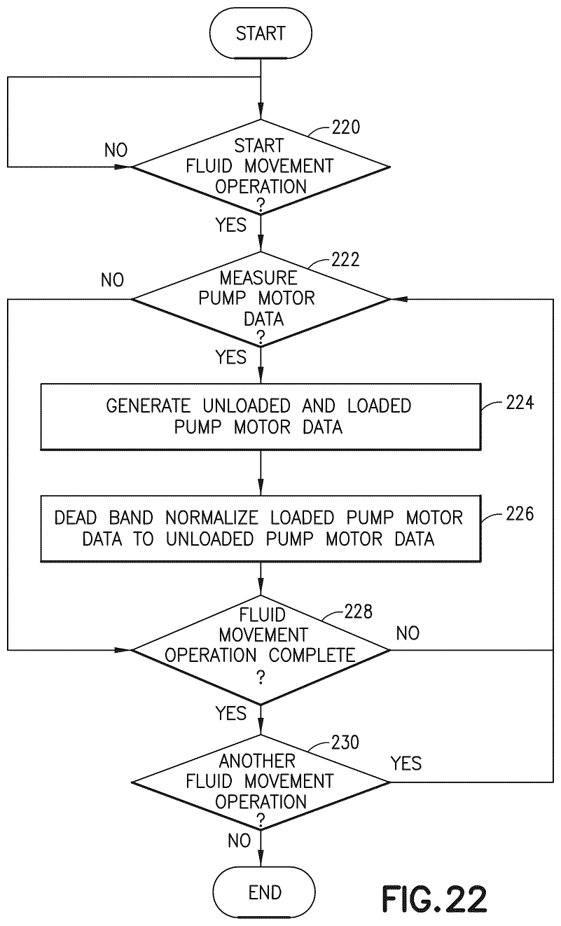

Operations associated with dead band normalization in accordance with illustrative embodiments of the technical solution described herein are shown in FIG. 22 and can be implemented, for example, as a dead band normalization algorithm performed by a controller (e.g., controller 192 in FIG. 6 of the afore-mentioned WO 2022/132555, or microcontroller 58 in FIG. 4 herein) or other device processing measured data. In accordance with an example embodiment, a controller for the fluid delivery device can be programmed or otherwise configured to obtain “loaded” measured data when drive mechanism is being operated for fluid movement (e.g., controlled intake or aspirating, or controlled output or dispensing), and normalize it with dead band or “unloaded” measured data obtained when the drive mechanism is being controlled for a fluid movement operation but momentarily is not moving fluid. In accordance with an example embodiment, dead band normalization is performed during an aspirate operation, a dispense operation, or during both types of operations. It can be beneficial to do it during fill and during delivery (e.g., disengage and reengage during any part of the overall aspirate or dispense stroke, depending on controlled volume intended to be drawn into the fluid chamber or delivered from fluid chamber). In any event, the dead band normalization data (e.g., the unloaded measured data) and the loaded measured data are optimally obtained during the same pump aspirate or dispense operation or stroke.

As illustrated in block 220 of FIG. 22, a pump controller can be configured, at the beginning of a fluid movement operation (e.g., an aspirate operation or stroke, or a dispense operation or stroke) to measure pump motor data related to that fluid movement operation (block 222). In accordance with an advantageous aspect of example embodiments of a technical solution described herein, the controller obtains or otherwise generates pump motor data comprising unloaded measured data and loaded measured data during that fluid movement operation (block 224). The controller performs dead band normalization in accordance with the technical solution described herein by normalizing the loaded measured data to the unloaded measured data corresponding to that fluid movement operation (block 226). It is to be understood that the dead band normalization can involve, for example, subtracting unloaded measured data from measured pump motor data to be able to advantageously determine fluid pressure or flow rate of the pump during that fluid delivery operation without being impacted by signal noise or noise factors (e.g., pump design and environmental factors). The unloaded measured data can be obtained at any point during the fluid movement operation wherein the drive mechanism does not move fluid. The loaded measured data can be obtained at multiple points during the fluid movement operation wherein the drive mechanism is engaged in moving fluid. In any event, the loaded and unloaded measured data obtained for that the fluid movement operation need not be used or relevant to a different fluid movement operation (block 230).

As stated before, the technical solution described herein successfully compensates for many changes in a pump (e.g., design factors of the battery, motor, and gearbox, and environment factors such as temperature) that are not related to changes in fluid pressure or flow rate or other measured parameter being used to detect occlusion or other condition of the fluid delivery device. An ideal normalization compensates for everything but pressure or flow rate, and this technical solution achieves essentially ideal normalization via dead band normalization illustrated in accordance with the example embodiments herein. As explained above with respect to the factors impacting motor current, for example, there are many terms and forces that ultimately add up to what current is measured, and the more of these terms or forces that can be normalized, the more accurate that occlusion detection using a measured parameter can be. Further, dead band normalizing as described herein allows an occlusion detection algorithm employing dead band normalization to evaluate individual fluid movement strokes or operations of a pump without having to look at changes over multiple strokes. Currently, there is no covering occlusion detection in infusion pumps related to current sensing or other measured pump motor parameter that utilizes a non-driving portion of the fluid movement to better assess fluid pressure or flow rate based on current or other measured motor parameter.

Occlusion in a fluid delivery device 10 such as an infusion pump for medication can result from restricted flow or pathway constriction such as a pinched catheter or tissue occlusion, or from an empty medication reservoir. It is important to measure fluid pressure or flow rate changes in the fluid delivery device from an occlusion or other pump malfunction for early detection to mitigate against possible fluid delivery inaccuracies resulting therefrom such as missed doses. The technical solution and example embodiments herein achieve more accurate and faster detection of occlusion and therefore fewer fluid delivery inaccuracies.

The measured data is indicative of pressure or flow rate and can be, but is not limited to, any of motor current, motor voltage, encoder count, motor drive count, delivery pulse energy, motor drive time, and so on. For example, current sensing is generally considered to be a reliable method of detecting occlusions in a fluid path of a fluid delivery device 10 because motor current can be indirectly correlated to pressure. As stated above, an occlusion causes a decrease in fluid flow in the fluid delivery device, which causes increased pressure. An increase in pressure causes an increase in torque demand required by the motor to overcome this pressure. The increase in torque demand corresponds to an increase in current drawn by the motor 66, which is one way to detect occlusions such as an occluded catheter, or air in the fluid path, or malfunction of the motor.

FIGS. 4-11 of the afore-mentioned WO 2022/132555 illustrate an example fluid delivery device having an example interface in a syringe-type fluid delivery mechanism that facilitates dead band normalization in accordance with example embodiments. In a syringe-type fluid delivery mechanism, a drive assembly can be selectively engaged and disengaged from the plunger to allow for operating the pump without fluid movement to obtain unloaded measured data for dead band normalization in accordance with example embodiments.

FIGS. 1 through 4 and 15 herein illustrate another example fluid delivery device having a different example interface from a syringe-type fluid delivery mechanism to facilitate dead band normalization in accordance with example embodiments. As explained below in connection with FIGS. 1-4 and 15, in a rotational metering-type fluid delivery mechanism, a gearbox and output gear coupling to a pump drive mechanism allows for the pump to be driven without moving fluid to obtain data in a dead band region 100 of an aspirate or dispense operation to obtain unloaded measured data for dead band normalization in accordance with example embodiments.

A gap between the piston 30 and the output gear 39 of the gearbox (e.g., between a tab 31 at the end of the piston and a slot 35 in the output gear 39) provides a beneficial interface for dead band normalization since it is close to a fluid driving interface that is capable at least temporarily of moving yet without moving the fluid during a fluid movement operation. For example, as illustrated in FIG. 20D, even when a drive mechanism operates a pump piston 30 in a pump aspirate or dispense operation that moves fluid, an initial point 100 after motor startup in a new direction is similar to unloaded measured data, and loaded measured data can be normalized to this unloaded measured data.

More specifically, FIG. 3D illustrates part of a manifold 22 having a motor and gearbox assembly 33 that cooperates with the pump assembly 20. The motor and gearbox assembly 33 includes an opening that can receive a rotational limit switch. In this manner, output gear 39, which is internal to the gearbox housing, can access and engage the flexures of a limit switch. Motor and gearbox assembly 33 also include an axial retention snap so that the pump assembly 20 may be snap-fit to the motor and gearbox assembly 33. Motor and gearbox 3 includes a rotational key within a pump-receiving socket to receive pump assembly 20 and prevent rotation of the pump assembly 20 relative to the motor and gearbox assembly 33. Output gear 39 includes a slot 35 adapted to receive a tab 31 provided on the piston 30. When assembled, tab 31 is received into slot 35 so that the output gear 39 can transmit torque to the piston 30. As the output gear 39 rotates, the pump piston tab 31 both rotates and slides axially in the slot 35 to provide a useful interface with which to obtain unloaded measured data for dead band normalization with loaded measured data corresponding to when the piston is moving fluid to or from the chamber 38. Metal spring flexures on the motor connections and limit switches are used to make electrical contact with pads on the circuit board 92 during final assembly.

Alternatively, an interface that can facilitate dead normalization in the example rotational metering-type infusion pump can be designed with respect to the helical groove 26 and coupling pin 28. During a discharge stroke, the piston 30 is turned in a first rotational direction and is driven along the helical path of the helical groove 26 in the sleeve 24 via the coupling pin 28. The pump piston 30 translates away from the gearbox while rotating, expelling fluid from the pump chamber 38 and out of the outlet port 46. During the discharge stroke, friction between the port seals and the outside diameter of the sleeve 24 is sufficient to ensure that the sleeve does not rotate during this portion of the pump cycle. During a valve state change after the discharge stroke, the coupling pin 28 reaches the distal end of helical groove 26 and torque continues to be transmitted from the output gear, to the pump piston 30, and to the sleeve 24 via the coupling pin 28. The sleeve 24 and pump piston 30 rotate as a unit with no relative axial motion. The side hole on the sleeve 24 moves from the outlet port 46 to the inlet port 44. During an intake stroke, the output gear turns the pump piston 30 and the piston is translated axially relative to the sleeve 24 due to interaction of the coupling pin 28 within the helical groove 26. The pump piston 30 translates toward the gearbox, pulling fluid from the reservoir into the pump chamber via the inlet port 44. During a valve state change after the intake stroke, the coupling pin 28 reaches the upper end of helical groove 26, the pump motor continues to deliver torque, causing the sleeve 24 and piston 30 to rotate together as a unit with no relative axial motion and the side hole of the sleeve 24 to move from alignment with the inlet port 44 to alignment with the outlet port 46. The helical groove 26 and coupling pin 28 can be configured by extending the groove or otherwise altering dimensions or slope of the groove to provide a dead region (e.g., 100 in FIG. 2) within the fluid movement operation wherein drive mechanism component(s) operate but do not move fluid to provide an interface for dead normalization.

In accordance with another example embodiment, a fluid delivery device can have a drive mechanism employing one or more cams that can provide a beneficial interface for dead band normalization in accordance with the present technical solution. Unloaded measured data for dead band normalization can be obtained, for example, using a dead region provided by a cam and cam follower. At some point during a fluid movement operation wherein an actuator with cam follower is being controlled to rotate relative to a cam, the cam follower's advancement along a flat surface of the cam does not result in a related gear or other component connected to the cam being operated to move fluid during that fluid movement operation.

Regardless of the type of actuator and drive mechanism 66 employed in a fluid delivery device 10 such as wearable medication infusion pump, dead band normalization advantageously uses an unloaded region or portion in a positive displacement pump fluid movement operation to obtain unloaded measured data related to fluid movement (e.g., pressure, flow rate, and so on) with which to normalize loaded measured data related to that fluid movement operation. The resulting normalized measured data is advantageous because signal noise related to the actuator and impact of external noise factors (e.g., environmental factors and part-to-part variation) is removed, allowing for more accurate occlusion detection using the normalized measured data. Another benefit of dead band normalization in accordance with the technical solution and example embodiments described herein is that the unloaded and loaded measured data signals used for dead band normalization are processed very locally, that is, close to a particular fluid movement event (e.g., a particular aspirate stroke or a dispense stroke). It is to be understood that this local or proximal operation is not limited by any particular timing or order of operation for obtaining the loaded and unloaded measured data during a particular fluid movement event or operation.

Reference will now be made to FIG. 23 which illustrates software operations of an example embodiment of an occlusion detection algorithm that can be performed by a controller associated with a fluid delivery device 10 such as a wearable patch pump, and works by monitoring the current drawn by the fluid delivery device 10's motor 66 and using this monitored current information to infer the pressure in the fluid delivery device 10's downstream fluid path. The current drawn by the fluid delivery device 10's motor 66 is measured, for example, by measuring the voltage across a resistor which is in series with the motor as shown in FIG. 15. This voltage value passes through a low-pass RC-filter on the fluid delivery device 10's printed circuit board 92 assembly (PCBA) and is then amplified before being passed through an analog-to-digital converter (ADC) provided on the PCBA. The voltage values output by the ADC are then used by calculations in the fluid delivery device's firmware to calculate the current passing through the resistor 142 and hence being drawn by the motor 66.

As stated above, the occlusion detection algorithm in FIG. 23 uses the motor current data measured to calculate two values for each dispense stroke performed. As indicated by blocks 250 and 252, the algorithm performs a pump cycle and increments a cycle counter. The first of these values is measuring motor current during a window or region of a stroke termed the “dead band” region 200 (i.e., the first window mentioned above in the overview for FIG. 23, as indicated at blocks 254 and 256. Current measured in the dead band region 200 corresponds to the motor current required to operate the fluid delivery device 10's gearbox 33 before the gearbox has engaged the pump's piston fin or tab 31. This dead band current value “iDeadband” can be used as a reference value for each stroke performed. Any increase in current drawn by the motor 66 from this dead band current value “iDeadband” can be attributed to additional torque acting on the motor, whereby this torque is created by the pump 20.

With reference to blocks 254, 258 and 280 in FIG. 23, the second value calculated by the occlusion detection algorithm in the example embodiment described with reference to FIG. 23 is the maximum current drawn by the motor in the window or region between the dead band region 200 and about 1.5 seconds into the stroke (i.e., the second window mentioned above in the overview for FIG. 23) for the rotational metering-type pump described in connection with FIGS. 1-4 and 15. This second window corresponds with the period of time in which the pump piston 30 is being driven forward during the dispense stroke. By taking the maximum current value “iPistonMax” in this second window of time during a dispense stroke, a measure of the “effort” required to drive the pump piston 30 forward is obtained. This value is dependent on the torque placed on the motor 66 due to the back pressure acting on the face of the piston 30, along with other forces and torques.

With continued reference to block 258 in FIG. 23, by subtracting iDeadband from iPistonMax, a value for the current required specifically to push the piston 30 forward is obtained. This value is referred to as “iStroke” and this value is used to estimate the pressure in the downstream fluid path of the fluid delivery device 10 once per stroke.

With reference to blocks 256, 258 and 280 in FIG. 23, the iStroke value is calculated in this manner for the first 50 dispense strokes performed by the fluid delivery device 10. In some instances the fluid delivery device 10's gearbox current or iDeadband value can decrease over time which contributes to the iStroke value climbing over time. To compensate for this decline in gearbox current or iDeadband value and its effect on the calculated iStroke value, a change is made to the iStroke calculation method after a selected number of dispense strokes (e.g., 50 delivery strokes). All strokes after selected number (e.g., 50) are calculated by measuring the iPistonMax for that stroke and subtracting the mean value of the first 50 deadband values measured during the delivery provided that the deadband value for the current stroke is not higher than the mean value of the first 50 deadband values. If the deadband value for a stroke is higher than the mean of the first 50 deadband values, then the fluid delivery device 10's occlusion detection algorithm reverts back to the original iStroke calculation method as used in the first 50 delivery strokes. These two iStroke calculation methods are summarized in the following equations:

$$For {iStroke}_{n = 1 \to 50}$$

$${iStroke}_{n = 1 \to 50} = Max Piston {Current}_{n} - Deadband {Current}_{n}$$

$$For {iStroke}_{n = 51 \to N}$$

$$if Deadband {Current}_{n} < mean ( {Deadband}_{n = 1 \to 50} )$$

$${iStroke}_{n = 51 \to N} = Max Piston {Current}_{n} - mean ( {Deadband}_{n = 1 \to 50} )$$

$$else$$

$${iStroke}_{n = 51 \to N} = Max Piston {Current}_{n} - Deadband {Current}_{n}$$

where n is the cycle number and N is the total number of strokes performed during an OBI device's delivery.

With reference to blocks 260 and 282 in FIG. 23, the fluid delivery device 10 calculates a baseline value using its initial results for the iStroke metric, as explained in further detail below. If the iStroke value calculated on any dispense stroke is lower than this baseline value, then the iStroke value for that stroke is taken to be the value of the device's baseline, i.e., the baseline value for a device is the lower limit of iStroke values for that device. Any iStroke values lower than the fluid delivery device 10's iBaseline value are rejected and replaced with iBaseline.

The occlusion detection algorithm then uses these iStroke values to perform logical operations and decide if the delivery should proceed or be halted.

With reference to blocks 260, 282, 262 and 284 in FIG. 23, the fluid delivery device 10 calculates the reference value iBaseline using the iStroke values from several (e.g., 4) activation strokes. These activation strokes can be performed during manufacturing of the fluid delivery device 10, for example. To calculate iBaseline, the fluid delivery device 10 calculates the mean value of all iStroke values that pass a screening test. To screen the iStroke values, the microcontroller 58 is programmed in accordance with the example occlusion detection algorithm in FIG. 23 to determine if each iStroke value is less than 1 mA different from the mean value of the other three strokes. If the iStroke value meets this criterion, then it is included in determining the iBaseline value; and if the iStroke value is greater than 1 mA from the mean value of the other three strokes, then it is deemed to be an outlier stroke and so it is not included in the calculation of iBaseline. If no iStroke values meet this criterion, then the value of iBaseline is set to 2.0 mA that has been selected as a mean of a plurality of fluid devices devices 10. It is to be understood that the 1 mA and 2.0 mA iBaseline values can be empirically determined for a plurality of a particular type of rotational metering-type or reciprocating-type pump and can be different values than 1 mA and 2.0 mA.

As used in block 268 in FIG. 23, the fluid delivery device 10 determines a threshold of iThreshold=iBaseline+iDelta_Thresh where iDelta_Thresh=3.85 mA. This value of 3.85 mA can be determined experimentally as allowing the device 10 to complete its delivery and not trigger an occlusion alarm while operating against the anticipated patient backpressures (e.g., 10 psi) while still allowing the device 10 to detect occlusions prior to leakage which would prevent this occlusion detection.

With reference to block 266 in FIG. 23, as the device 10 performs its dispense strokes, it calculates the change in iStroke from the previous value by subtracting the previous cycle's iStroke value from the most recently completed cycle. For the first pump cycle, the iBaseline value can be used as the previous cycle value of iStroke). This change in iStroke parameter is referred to as “Delta_iStroke”. The Delta_iStroke value is calculated using the same method throughout all strokes and is not changed after 50 strokes:

$${Delta\_iStroke}_{n} = ( Max Piston {Current}_{n} - Deadband {Current}_{n} ) - ( Max Piston {Current}_{n - 1} - Deadband {Current}_{n - 1} )$$

A threshold value for this Delta iStroke metric is set in the firmware at 0.9 mA and is referred to as the “RapidRise” parameter. The 0.9 mA value for RapidRise can be determined from experimental data that shows that this level of Delta_iStroke typically does not occur in devices 10 that are not occluded.

With reference to blocks 266 and 268 in FIG. 23, the fluid delivery device 10 can use the iStroke and Delta iStroke metrics to assess whether an occlusion has occurred. On each stroke, the algorithm in FIG. 23 performs two checks: (1) the algorithm checks if the Delta iStroke value exceeds 0.9 mA, and (2) the algorithm checks if the iStroke value exceeds iThreshold. If either of these metrics exceeds its corresponding threshold value, then an occlusion warning flag is generated as indicated at block 270.

In addition to the two metrics related to motor current described with reference to blocks 266 and 268, the fluid delivery device 10 can also use the time taken to close and release its end-stop switch 90, as indicated at block 264. As described above in connection with FIGS. 3C and 9, this end-stop switch 90 is closed by the pump's sleeve rotating from the dispense side to the aspirate side. Under normal operation the end-stop switch is closed for less than 0.5 seconds; however, some occlusions cause the pump to prematurely rotate and, when this occurs, the end-stop duration is closer to ˜1.75 seconds. This change in end-stop duration is a reliable way of detecting this premature rotation behavior. Therefore, the occlusion detection algorithm in FIG. 23 checks the end-stop duration and, if it exceeds 1 second, then an occlusion warning flag is generated.

If the fluid delivery device 10 generates any of these occlusion flags on two successive strokes, then the device 10's occlusion alarm is triggered, as indicated in blocks 272, 274 and 276. An occlusion alarm causes the device 10's motor 66 to stop, causes the device 10 to sound a repeating tone from its buzzer, and/or causes the device 10's LEDs to flash repeatedly. The design to have the device 10 detect two successive strokes is based on test data showing that the device 10 can reach a leaking state after two strokes in occlusions with minimal compliance. More typically, it takes more strokes for a fluid delivery device 10 as shown in FIGS. 1-4 to reach pressures where a gross leak occurs due to compliance of the device 10's plastic components and air entrapped in the fluid path. Therefore, if such a fluid delivery device 10 increases in pressure very suddenly and reaches a leak state in only two strokes, the device 10 will still be able to deactivate itself as the second of these occluded strokes will result in occlusion flags being raised. Requiring two strokes successively to trigger an occlusion also reduces the risk of a false positive alarm being triggered due to a non-pressure noise factor causing a single stroke to have an excessively high iStroke value.

With reference to block 286 in FIG. 23, if no occlusion signatures are detected per blocks 264, 266 and 268, then the fluid delivery device 10 sets an occlusion counter to 0 (block 288) and continues to perform another pump cycle as indicated at blocks 278 and 250. If a total cycle counter (e.g., 240) is reached or the reservoir is empty, or the desired amount of fluid to be delivered has in fact been delivered, per block 290, then the fluid delivery performed by the device 10 can be indicated as complete per block 292.

It will be understood by one skilled in the art that this disclosure is not limited in its application to the details of construction and the arrangement of components set forth in the foregoing description or illustrated in the drawings.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., erasable programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A wearable infusion patch pump comprising:
a pump comprising a chamber configured with at least one port to receive fluid into the chamber from a reservoir and to pass the fluid out of the chamber, and a pumping mechanism comprising a gearbox assembly operated by a motor to extend a piston in the chamber to control dispensing of a volume of the fluid from the chamber during a dispense stroke;
a current sensing device configured to detect motor current corresponding to current drawn by the motor during each said dispense stroke performed by the pumping mechanism among a plurality of dispense strokes; and
a processor, and for each of the plurality of dispense strokes_{n=1, . . . , x} performed by the pumping mechanism, the processor is operated in accordance with programmed instructions to:
a) determine values from the motor current corresponding to said dispense strokeₙ chosen from a dead band value (iDeadbandₙ) and a maximum piston current value (iPistonMaxₙ), the dead band value (iDeadbandₙ) corresponding to a current drawn by the motor when the pumping mechanism is moving but without causing fluid to flow with respect to the chamber such that an increase in the motor current over the dead band value (iDeadbandₙ) corresponds to a torque acting on the motor when the piston in the pumping mechanism moves against the fluid in the chamber during said dispense stroke, and the maximum piston current value (iPistonMaxₙ) corresponding to a maximum amount of current drawn by the motor during a portion of said dispense stroke wherein the piston moves against the fluid in the chamber to dispense the fluid from the at least one port;
b) determine a stroke current value (iStrokeₙ) for said dispense stroke by subtracting the dead band value (iDeadbandₙ) from the maximum piston current value (iPistonMaxₙ) corresponding to the said dispense strokeₙ;
c) determine a difference (Delta_iStroke) between the stroke current value (iStrokeₙ) and a previous stroke current value (iStrokeₙ₋₁) determined for a previous one of the plurality of dispense stokes,
d) increment a counter when at least one occlusion condition is detected, the at least one occlusion condition comprising the difference (Delta_iStroke) exceeding a rapid rise threshold (iRapidRise) having a designated value corresponding to an increase in the motor current that indicates an unwanted occlusion occurring in the pump; and
e) terminating dispensing of the fluid from the chamber when the counter reaches a value T≥2.

2. The wearable infusion patch pump of claim 1, wherein the current sensing device comprises a resistor connected in series with the motor, and the processor being configured to calculate the motor current using a voltage measured across the resistor.

3. The wearable infusion patch pump of claim 1, wherein said dispense stroke is characterized by a stroke duration comprising a first time period corresponding to a dead band period during which the pumping mechanism is moving but without causing fluid to flow with respect to the chamber, and a second time period that follows the first time period and during which the pumping mechanism can cause the fluid to flow from the chamber.

4. The wearable infusion patch pump of claim 3, wherein the processor determines the dead band value (iDeadbandₙ) from calculating the motor current using a voltage measured across the resistor during the first time period.

5. The wearable infusion patch pump of claim 3, wherein the processor determines the maximum piston current value (iPistonMaxₙ) from calculating the motor current using a voltage measured across the resistor during the second time period.

6. The wearable infusion patch pump of claim 5, wherein the processor determines the maximum piston current value (iPistonMaxₙ) from calculating a maximum value of the motor current during a beginning portion of the second time period.

7. The wearable infusion patch pump of claim 1, wherein the pumping mechanism is configured to operate for at least a designated total number (X) of dispense strokes_{n=1, . . . , x} to deliver the fluid from the chamber, and the stroke current value (iStrokeₙ) is determined using a first calculation during a designated initial number (n=1, . . . , Y) of the dispense strokes, and is determined using a second calculation different from the first calculation during a remaining number (n=Y+1, . . . , X) of the dispense strokes.

8. The wearable infusion patch pump of claim 7, wherein the first calculation corresponds to the processor subtracting the dead band value (iDeadbandₙ) from the maximum piston current value (iPistonMaxₙ) corresponding to said dispense stroke_{n=1, . . . , Y}.

9. The wearable infusion patch pump of claim 7, wherein the second calculation comprises the processor determining a mean value of the dead band value (iDeadbandₙ) for each of the designated initial number (n=1, . . . , Y) of the dispense strokes, and using the first calculation when the dead band value for the dispense stroke is greater than or equal to the mean value and using the second calculation when the dead band value for the dispense stroke is less than the mean value, the second calculation corresponding to subtracting the mean value from the maximum piston current value corresponding to the said dispense stroke_{n=Y+1, . . . , X}.

10. The wearable infusion patch pump of claim 1, wherein the processor is further configured to operate in accordance with programmed instructions to determine a current baseline value (iBaseline), and to use the current baseline value (iBaseline) instead of the stroke current value (iStrokeₙ) for the said dispense stroke to determine the difference (Delta_iStroke) when the stroke current value (iStrokeₙ) is less than the baseline value (iBaseline).

11. The wearable infusion patch pump of claim 10, wherein the processor is further configured to operate in accordance with programmed instructions to determine the baseline value (iBaseline) during an initial operation related to the pump chosen from manufacturing of the patch pump, assembling the patch pump, initializing the patch pump, and a priming operation by the pump.

12. The wearable infusion patch pump of claim 11, wherein the processor is further configured to operate in accordance with programmed instructions to calculate the baseline value (iBaseline) using a mean value of motor current corresponding to a predetermined number of said dispense stroke previously occurring during the initial operation and precluding the motor current from the mean value when the motor current differs from the mean value by a designated amount.

13. The wearable infusion patch pump of claim 10, wherein the processor is further configured to operate in accordance with programmed instructions to:
determine an acceptable backpressure threshold value (iDelta_Threshold) that corresponds to the motor current occurring while the piston is operating against patient-related backpressure that occurs when the pump is delivering the fluid into a patient and is less than an unwanted occlusion being detected by the processor, wherein the at least one occlusion condition further comprises the stroke current value (iStrokeₙ) exceeding an occlusion threshold value (iThreshold) corresponding to a sum of the current baseline value (iBaseline) and the acceptable backpressure threshold value (iDelta_Threshold).

14. The wearable infusion patch pump of claim 1, wherein the rapid rise threshold (iRapidRise) is determined experimentally during operation of the pump to be a value of the motor current at which an unwanted occlusion is not occurring in the pump.
